(19) United States
(12) Patent Application Publication
(10) Pub. No.: US 2014/0271621 A1
(43) Pub. Date: Sep. 18, 2014

(54) METHODS OF PROGNOSIS AND DIAGNOSIS OF PANCREATIC CANCER

(71) Applicant: Abbott Laboratories, Abbott Park, IL (US)

(72) Inventors: Philip M. Hemken, Pleasant Prairie, WI (US); Barry L. Dowell, Mundelein, IL (US); Gerard J. Davis, Wauconda, IL (US)

(21) Appl. No.: 13/828,319

(22) Filed: Mar. 14, 2013

(51) Int. Cl. G01N 33/574

(52) U.S. Cl. CPC G01N 33/57438
USPC 424/133.1; 435/7.23

(57) ABSTRACT

Disclosed herein are methods of diagnosing pancreatic cancer in a patient by detecting the presence and/or amount of at least three biomarkers of pancreatic cancer in a sample from the patient. The methods and biomarkers may be used to develop an accurate prognosis for a patient having cancer or suspected of having pancreatic cancer, or to accurately diagnose a patient having, or suspected of having pancreatic cancer. The methods and biomarkers may be used to identify and/or classify a patient as a candidate for a cancer therapy.

### TECHNICAL FIELD

The disclosure relates to methods and immunoassay platforms for determining a prognosis, diagnosis, or risk identification of pancreatic cancer in a patient by detecting one or more biomarkers in the patient as well as determining amounts thereof. The biomarkers may be used to identify a patient with pancreatic cancer, identify a patient as a candidate for pancreatic cancer therapy, to classify a patient's risk of developing pancreatic cancer, or to classify a patient's cancer stage or risk of progression of cancer, as well as to determine a diagnosis, prognosis, or a treatment regimen.

### BACKGROUND

Despite medical advances, pancreatic cancer is one of the most deadly diseases. In 2011, there were estimated 44,030 people diagnosed with pancreatic cancer in the U.S. of which approximately 37,660 people died. Pancreatic cancer is the fourth most common cancer-related cause of death among U.S. men (after lung, prostate and colorectal cancer) and women (after lung, breast and colorectal cancer). The peak incidence occurs in the 7th and 8th decades of life with the incidence nearly the same in both men and women. While death rates of lung, colorectal, breast, and prostate cancer have declined in the U.S. since 2003, pancreatic cancer has increased during that same time. The estimated new cases of pancreatic cancer globally in 2011 in developed countries is 84,200 (men) and 80,900 (women), while the estimated deaths are 82,700 (men) and 79,100 (women). Unfortunately, most of the symptomatic patients are incurable. The prognosis for pancreatic cancer patients is very poor with a 5-year relative survival rate of 6% for all stages combined. This is due to the late stage of the disease at the time of diagnosis.

There is a great need for early detection of pancreatic cancer to improve the survival rate of these patients. Some experts recommend that screening should be implemented with high risk individuals with inexpensive, and noninvasive diagnostic tests for early lesions. These high risk individuals include patients with pancreatitis, pancreatic intraepithelial neoplasias [PanINs] or intraductal papillary mucinous neoplasms [IPMNs]). Serum biomarker panels may provide a means for screening these targeted high risk individuals, which could greatly increase the survival rates.

### SUMMARY

The present invention is directed to a method for identifying and treating a subject having or at risk of having pancreatic cancer, the method comprising the steps of obtaining a biological sample from the subject; determining the levels of carbohydrate antigen 19-9 (CA19-9), carbohydrate antigen 125 (CA125), and CUB and zona pellucida-like domains 1 (CUZD1) in the biological sample from the subject; comparing the levels of CA19-9, CA125, and CUZD1 in the biological sample to reference levels of CA19-9, CA125, and CUZD1; identifying the subject as having pancreatic cancer or at risk of having pancreatic cancer if the levels of CA19-9, CA125, and CUZD1 in the biological sample are greater than the reference levels of CA19-9, CA125, and CUZD1; and administering a therapy against pancreatic cancer to the subject identified as having pancreatic cancer or a means by which to monitor pancreatic status in said subject identified as at risk of having pancreatic cancer. The method further comprises a step of administering a means for confirming the subject has pancreatic cancer by performing a biopsy, measuring pancreatic function, or imaging with MRI, PET scan, CT scan, ultrasound, endoscopic ultrasound (EUS), endoscopic retrograde cholangiopancreatography (ERCP), EUS cholangiopancreatography, MRI/magnetic resonance cholangiopancreatography (MRI/MRCP) or laparoscopy. The reference levels of CA19-9, CA125, and CUZD1 are the levels of CA19-9, CA125, and CUZD1 in a control sample. The control sample is a sample from a benign or healthy patient. The reference levels of CA19-9, CA125, and CUZD1 are the CA19-9, CA125, and CUZD1 cutoff values determined by a receiver operating curve (ROC) analysis from biological samples of a patient group. The reference levels of CA19-9, CA125, and CUZD1 are the CA19-9, CA125, and CUZD1 cutoff values determined by a quartile analysis of biological samples of a patient group. The CA19-9, CA125, and CUZD1 cutoff values are about 20 ng/mL in serum for CA19-9, about 15 ng/mL for CA125, and about 1.5 ng/mL in serum for CUZD1. The CA19-9, CA125, and CUZD1 levels in the biological sample above the cutoffs indicate that the subject has early stage pancreatic cancer or late stage pancreatic cancer. The subject may have stage I pancreatic cancer. The method further comprises determining the level of at least one additional biomarker of pancreatic cancer in the biological sample, and comparing the level of the at least one additional biomarker of pancreatic cancer to a reference concentration value for the at least one biomarker of pancreatic cancer. The at least one additional biomarker of pancreatic cancer is selected from the group consisting of CEA, HE4, NGAL, TIMP-1, CA15-3, AGR2, LRG1, COL6A3, REG1B and SYCN. The subject may be a human. The biological sample of a subject may be selected from a tissue sample, bodily fluid, whole blood, plasma, serum, urine, bronchoalveolar lavage fluid, and a cell culture suspension or fraction thereof. Determining the levels of CA19-9, CA125, and CUZD1 comprises an immunological method with molecules binding to CA19-9, CA125, and CUZD1. The molecules binding to CA19-9, CA125, and CUZD1 comprises at least one antibody capable of specifically binding CA19-9, CA125, or CUZD1. The immunological method comprises measuring the levels of CA19-9 by contacting the test sample with at least one capture antibody, wherein the capture antibody binds to an epitope on CA19-9 or a fragment of CA19-9 to form a capture antibody-CA19-9 antigen complex; contacting the capture antibody-CA19-9 antigen complex with at least one detection antibody comprising a detectable label, wherein the detection antibody binds to an epitope on CA19-9 that is not bound by the capture antibody and forms a capture antibody-CA19-9 antigen-detection antibody complex; and determining the CA19-9 levels in the test sample based on the signal generated by the detectable label in the capture antibody-CA19-9 antigen-detection antibody complex formed in the previous step; measuring the levels of CA125 by contacting the test sample with at least one capture antibody, wherein the capture antibody binds to an epitope on CA125 or a fragment of CA125 to form a capture antibody-CA125 antigen complex; contacting the capture antibody-CA125 antigen complex with at least one detection antibody comprising a detectable label, wherein the detection antibody binds to an epitope on CA125 that is not bound by the capture antibody and forms a capture antibody-CA125 antigen-detection antibody complex; and determining the CA125 levels in the test sample based on the signal generated by the detectable label in the capture antibody-CA125 antigen-detection antibody complex formed in the previous step; and measuring the levels of CUZD1 by contacting the test sample with at least one capture antibody, wherein the capture antibody binds to an epitope on CUZD1 or a fragment of CUZD1 to form a capture antibody-CUZD1 antigen complex; contacting the capture antibody-CUZD1 antigen complex with at least one detection antibody comprising a detectable label, wherein the detection antibody binds to an epitope on CUZD1 that is not bound by the capture antibody and forms a capture antibody-CUZD1 antigen-detection antibody complex; and determining the CUZD1 levels in the test sample based on the signal generated by the detectable label in the capture antibody-CUZD1 antigen-detection antibody complex formed in the previous step. The therapy against pancreatic cancer comprises administering at least one of surgery, radiation therapy, chemotherapy, and targeted therapy to the subject. The means by which to monitor pancreatic status comprises at least one of determining the levels of at least one biomarker of pancreatic cancer at periodic intervals, performing a biopsy, measuring pancreatic function, or imaging with MRI, PET scan, CT scan, ultrasound, endoscopic ultrasound (EUS), endoscopic retrograde cholangiopancreatography (ERCP), EUS cholangiopancreatography, MRI/magnetic resonance cholangiopancreatography (MRI/MRCP) or laparoscopy.

The present invention is also directed to a method for the diagnosis, prognosis and/or risk stratification of pancreatic cancer in a subject having or suspected of pancreatic cancer, the method comprising the step of detecting increased levels of carbohydrate antigen 19-9 (CA19-9), carbohydrate antigen 125 (CA125), and CUB and zona pellucida-like domains 1 (CUZD1) in the subject relative to a control subject not having pancreatic cancer. The method further comprises a step of administering a means for confirming the diagnosis, prognosis and/or risk stratification of pancreatic cancer in a subject by performing a biopsy, measuring pancreatic function, or imaging with MRI, PET scan, CT scan, ultrasound, endoscopic ultrasound (EUS), endoscopic retrograde cholangiopancreatography (ERCP), EUS cholangiopancreatography, MRI/magnetic resonance cholangiopancreatography (MRI/MRCP) or laparoscopy. The method further comprises determining the level of at least one additional biomarker of pancreatic cancer in the biological sample, and comparing the level of the at least one additional biomarker of pancreatic cancer to a reference concentration value for the at least one biomarker of pancreatic cancer. The at least one additional biomarker of pancreatic cancer is selected from the group consisting of CEA, HE4, NGAL, TIMP-1, CA15-3, AGR2, LRG1, COL6A3, REG1B and SYCN. The subject is a human. The biological sample of a subject is selected from a tissue sample, bodily fluid, whole blood, plasma, serum, urine, bronchoalveolar lavage fluid, and a cell culture suspension or fraction thereof. Determining the levels of CA19-9, CA125, and CUZD1 comprises an immunological method with molecules binding to CA19-9, CA125, and CUZD1. The molecules binding to CA19-9, CA125, and CUZD1 comprises at least one antibody capable of specifically binding CA19-9, CA125, or CUZD1. The immunological method comprises measuring the levels of CA19-9 by contacting the test sample with at least one capture antibody, wherein the capture antibody binds to an epitope on CA19-9 or a fragment of CA19-9 to form a capture antibody-CA19-9 antigen complex; contacting the capture antibody-CA19-9 antigen complex with at least one detection antibody comprising a detectable label, wherein the detection antibody binds to an epitope on CA19-9 that is not bound by the capture antibody and forms a capture antibody-CA19-9 antigen-detection antibody complex; and determining the CA19-9 levels in the test sample based on the signal generated by the detectable label in the capture antibody-CA19-9 antigen-detection antibody complex formed in the previous step; measuring the levels of CA125 by contacting the test sample with at least one capture antibody, wherein the capture antibody binds to an epitope on CA125 or a fragment of CA125 to form a capture antibody-CA125 antigen complex; contacting the capture antibody-CA125 antigen complex with at least one detection antibody comprising a detectable label, wherein the detection antibody binds to an epitope on CA125 that is not bound by the capture antibody and forms a capture antibody-CA125 antigen-detection antibody complex; and determining the CA125 levels in the test sample based on the signal generated by the detectable label in the capture antibody-CA125 antigen-detection antibody complex formed in the previous step; and measuring the levels of CUZD1 by contacting the test sample with at least one capture antibody, wherein the capture antibody binds to an epitope on CUZD1 or a fragment of CUZD1 to form a capture antibody-CUZD1 antigen complex; contacting the capture antibody-CUZD1 antigen complex with at least one detection antibody comprising a detectable label, wherein the detection antibody binds to an epitope on CUZD1 that is not bound by the capture antibody and forms a capture antibody-CUZD1 antigen-detection antibody complex; and determining the CUZD1 levels in the test sample based on the signal generated by the detectable label in the capture antibody-CUZD1 antigen-detection antibody complex formed in the previous step. The method further comprises a step of administering at least one of a therapy against pancreatic cancer to the subject or a means by which to monitor pancreatic status in the subject.

The present invention is also directed to a method of providing a diagnosis of a subject having pancreatic cancer, the method comprising the steps of obtaining a biological sample comprising blood from the subject; determining the level of carbohydrate antigen 19-9 (CA19-9), carbohydrate antigen 125 (CA125), and CUB and zona pellucida-like domains 1 (CUZD1) in the biological sample from the subject; comparing the levels of CA19-9, CA125, and CUZD1 in the biological sample to reference levels of CA19-9, CA125, and CUZD1; and providing a diagnosis of a subject having pancreatic cancer if the levels of CA19-9, CA125, and CUZD1 in the biological sample are greater than the reference levels of CA19-9, CA125, and CUZD1. The method further comprises a step of administering a means for confirming the subject has pancreatic cancer by performing a biopsy, measuring pancreatic function, or imaging with MRI, PET scan, CT scan, ultrasound, endoscopic ultrasound (EUS), endoscopic retrograde cholangiopancreatography (ERCP), EUS cholangiopancreatography, MRI/magnetic resonance cholangiopancreatography (MRI/MRCP) or laparoscopy. The reference levels of CA19-9, CA125, and CUZD1 are the levels of CA19-9, CA125, and CUZD1 in a control sample. The control sample is a sample from benign or normal patients. The reference levels of CA19-9, CA125, and CUZD1 are the CA19-9, CA125, and CUZD1 cutoff values determined by a receiver operating curve (ROC) analysis from biological samples of a patient group. The reference levels of CA19-9, CA125, and CUZD1 are the CA19-9, CA125, and CUZD1 cutoff values determined by a quartile analysis of biological samples of a patient group. The CA19-9, CA125, and CUZD1 cutoff values are about 20 ng/mL in serum for CA19-9, about 15 ng/mL for CA125, and about 1.5 ng/mL in serum for CUZD1. The CA19-9, CA125, and CUZD1 levels in the biological sample above the cutoffs indicate that the subject has early stage pancreatic cancer or late stage pancreatic cancer. The subject may have stage I pancreatic cancer. The method further comprises determining the level of at least one additional biomarker of pancreatic cancer in the biological sample, and comparing the level of the at least one additional biomarker of pancreatic cancer to a reference concentration value for the at least one biomarker of pancreatic cancer. The at least one additional biomarker of pancreatic cancer is selected from the group consisting of CEA, HE4, NGAL, TIMP-1, CA15-3, AGR2, LRG1, COL6A3, REG1B and SYCN. The subject is a human. The biological sample of a subject is selected from a tissue sample, bodily fluid, whole blood, plasma, serum, urine, bronchoalveolar lavage fluid, and a cell culture suspension or fraction thereof. Determining the levels of CA19-9, CA125, and CUZD1 comprises an immunological method with molecules binding to CA19-9, CA125, and CUZD1. The molecules binding to CA19-9, CA125, and CUZD1 comprises at least one antibody capable of specifically binding CA19-9, CA125, or CUZD1. The immunological method comprises measuring the levels of CA19-9 by contacting the test sample with at least one capture antibody, wherein the capture antibody binds to an epitope on CA19-9 or a fragment of CA19-9 to form a capture antibody-CA19-9 antigen complex; contacting the capture antibody-CA19-9 antigen complex with at least one detection antibody comprising a detectable label, wherein the detection antibody binds to an epitope on CA19-9 that is not bound by the capture antibody and forms a capture antibody-CA19-9 antigen-detection antibody complex; and determining the CA19-9 levels in the test sample based on the signal generated by the detectable label in the capture antibody-CA19-9 antigen-detection antibody complex formed in the previous step; measuring the levels of CA125 by contacting the test sample with at least one capture antibody, wherein the capture antibody binds to an epitope on CA125 or a fragment of CA125 to form a capture antibody-CA125 antigen complex; contacting the capture antibody-CA125 antigen complex with at least one detection antibody comprising a detectable label, wherein the detection antibody binds to an epitope on CA125 that is not bound by the capture antibody and forms a capture antibody-CA125 antigen-detection antibody complex; and determining the CA125 levels in the test sample based on the signal generated by the detectable label in the capture antibody-CA125 antigen-detection antibody complex formed in the previous step; and measuring the levels of CUZD1 by contacting the test sample with at least one capture antibody, wherein the capture antibody binds to an epitope on CUZD1 or a fragment of CUZD1 to form a capture antibody-CUZD1 antigen complex; contacting the capture antibody-CUZD1 antigen complex with at least one detection antibody comprising a detectable label, wherein the detection antibody binds to an epitope on CUZD1 that is not bound by the capture antibody and forms a capture antibody-CUZD1 antigen-detection antibody complex; and determining the CUZD1 levels in the test sample based on the signal generated by the detectable label in the capture antibody-CUZD1 antigen-detection antibody complex formed in the previous step. The method further comprises a step of administering at least one of a therapy against pancreatic cancer to the subject or a means by which to monitor pancreatic status in the subject.

The present invention is also directed to method of determining whether a subject is suffering from early or late stage pancreatic cancer, the method comprising the steps of obtaining a biological sample comprising blood from the subject; determining the level of carbohydrate antigen 19-9 (CA19-9), carbohydrate antigen 125 (CA125), and CUB and zona pellucida-like domains 1 (CUZD1) in the biological sample from the subject; comparing the levels of CA19-9, CA125, and CUZD1 in the biological sample to reference levels of CA19-9, CA125, and CUZD1; and providing a diagnosis of a subject suffering from early stage or late stage pancreatic cancer if the levels of CA19-9, CA125, and CUZD1 in the biological sample are greater than the reference levels of CA19-9, CA125, and CUZD1. The method further comprises a step of administering a means for confirming the subject is suffering from early stage or late stage pancreatic cancer by performing a biopsy, measuring pancreatic function, or imaging with MRI, PET scan, CT scan, ultrasound, endoscopic ultrasound (EUS), endoscopic retrograde cholangiopancreatography (ERCP), EUS cholangiopancreatography, MRI/magnetic resonance cholangiopancreatography (MRI/MRCP) or laparoscopy. The reference levels of CA19-9, CA125, and CUZD1 are the levels of CA19-9, CA125, and CUZD1 in a control sample. The control sample is a sample from benign or normal patients. The control sample is a sample from benign or normal patients. The reference levels of CA19-9, CA125, and CUZD1 are the CA19-9, CA125, and CUZD1 cutoff values determined by a receiver operating curve (ROC) analysis from biological samples of a patient group. The reference levels of CA19-9, CA125, and CUZD1 are the CA19-9, CA125, and CUZD1 cutoff values determined by a quartile analysis of biological samples of a patient group. The CA19-9, CA125, and CUZD1 cutoff values are about 20 ng/mL in serum for CA19-9, about 15 ng/mL for CA125, and about 1.5 ng/mL in serum for CUZD1. The CA19-9, CA125, and CUZD1 levels in the biological sample above the cutoffs indicate that the subject has early stage pancreatic cancer or late stage pancreatic cancer. The subject may have stage I pancreatic cancer. The method further comprises determining the level of at least one additional biomarker of pancreatic cancer in the biological sample, and comparing the level of the at least one additional biomarker of pancreatic cancer to a reference concentration value for the at least one biomarker of pancreatic cancer. The at least one additional biomarker of pancreatic cancer is selected from the group consisting of CEA, HE4, NGAL, TIMP-1, CA15-3, AGR2, LRG1, COL6A3, REG1B and SYCN. The subject is a human. The biological sample of a subject is selected from a tissue sample, bodily fluid, whole blood, plasma, serum, urine, bronchoalveolar lavage fluid, and a cell culture suspension or fraction thereof. The determining the levels of CA19-9, CA125, and CUZD1 comprises an immunological method with molecules binding to CA19-9, CA125, and CUZD1. The molecules binding to CA19-9, CA125, and CUZD1 comprises at least one antibody capable of specifically binding CA19-9, CA125, or CUZD1. The immunological method comprises measuring the levels of CA19-9 by contacting the test sample with at least one capture antibody, wherein the capture antibody binds to an epitope on CA19-9 or a fragment of CA19-9 to form a capture antibody-CA19-9 antigen complex; contacting the capture antibody-CA19-9 antigen complex with at least one detection antibody comprising a detectable label, wherein the detection antibody binds to an epitope on CA19-9 that is not bound by the capture antibody and forms a capture antibody-CA19-9 antigen-detection antibody complex; and determining the CA19-9 levels in the test sample based on the signal generated by the detectable label in the capture antibody-CA19-9 antigen-detection antibody complex formed in the previous step; measuring the levels of CA125 by contacting the test sample with at least one capture antibody, wherein the capture antibody binds to an epitope on CA125 or a fragment of CA125 to form a capture antibody-CA125 antigen complex; contacting the capture antibody-CA125 antigen complex with at least one detection antibody comprising a detectable label, wherein the detection antibody binds to an epitope on CA125 that is not bound by the capture antibody and forms a capture antibody-CA125 antigen-detection antibody complex; and determining the CA125 levels in the test sample based on the signal generated by the detectable label in the capture antibody-CA125 antigen-detection antibody complex formed in the previous step; and measuring the levels of CUZD1 by contacting the test sample with at least one capture antibody, wherein the capture antibody binds to an epitope on CUZD1 or a fragment of CUZD1 to form a capture antibody-CUZD1 antigen complex; contacting the capture antibody-CUZD1 antigen complex with at least one detection antibody comprising a detectable label, wherein the detection antibody binds to an epitope on CUZD1 that is not bound by the capture antibody and forms a capture antibody-CUZD1 antigen-detection antibody complex; and determining the CUZD1 levels in the test sample based on the signal generated by the detectable label in the capture antibody-CUZD1 antigen-detection antibody complex formed in the previous step. The method further comprises a step of administering at least one of a therapy against pancreatic cancer to the subject or a means by which to monitor pancreatic status in the subject.

The present invention is also directed to a method of monitoring the progression of pancreatic cancer in a subject, the method comprising obtaining a biological sample from the subject determining the levels of carbohydrate antigen 19-9 (CA19-9), carbohydrate antigen 125 (CA125), and CUB and zona pellucida-like domains 1 (CUZD1) in the biological sample from the subject; correlating the levels of CA19-9, CA125, and CUZD1 with progression of pancreatic cancer in the subject, wherein if the levels of CA19-9, CA125, and CUZD1 are higher as compared to the levels of CA19-9, CA125, and CUZD1 in an earlier biological sample from the subject, the subject is identified as having progression of pancreatic cancer.

The present invention is also directed to a method of determining the stage of pancreatic cancer in a subject, the method comprising the steps of obtaining a biological sample comprising blood from the subject; determining the level of carbohydrate antigen 19-9 (CA19-9), carbohydrate antigen 125 (CA125), and CUB and zona pellucida-like domains 1 (CUZD1) in the biological sample from the subject; comparing the levels of CA19-9, CA125, and CUZD1 in the biological sample to first reference levels of CA19-9, CA125, and CUZD1 and second reference levels of CA19-9, CA125, and CUZD1; and correlating the levels of CA19-9, CA125, and CUZD1 in the biological sample with the stage of pancreatic cancer in the subject, wherein if the levels of CA19-9, CA125, and CUZD1 are greater than the first reference levels of CA19-9, CA125, and CUZD1 but less than the second reference levels of CA19-9, CA125, and CUZD1, the subject is determined to have early stage pancreatic cancer, and wherein if the levels of CA19-9, CA125, and CUZD1 are greater than the first reference levels of CA19-9, CA125, and CUZD1 and the second reference levels of CA19-9, CA125, and CUZD1, the subject is determined to have late stage pancreatic cancer. The method further comprises a step of administering at least one of a therapy against pancreatic cancer to the subject or a means by which to monitor pancreatic status in the subject.

The present invention is also directed to a kit for performing the method as described above, the kit comprising at least one reagent capable of specifically binding CA19-9, CA125, or CUZD1 to quantify the levels of CA19-9, CA125, or CUZD1 in the biological sample of a subject; and a reference standard indicating reference levels of CA19-9, CA125, and CUZD1. The at least one reagent comprises at least one antibody capable of specifically binding CA19-9, CA125, or CUZD1. The kit further comprises at least one additional reagent capable of binding at least one additional biomarker of CA19-9, CA125, and CUZD1 in the biological sample to quantify the concentration of the at least one additional biomarker in the biological sample, and a reference standard indicating a reference concentration of the at least one additional biomarker of CA19-9, CA125, and CUZD1 in the biological sample.

### BRIEF DESCRIPTION OF THE DRAWINGS

FIG. 1 shows an example of ARCHITECT® sandwich immunoassay.

FIG. 2 shows a schematic diagram of an ELISA-sandwich assay.

FIG. 3 shows a dot plot of the ARCHITECT® CA19-9 XR assay results.

FIG. 4 shows a dot plot of the ARCHITECT® CA-125 II assay results.

FIG. 5 shows a dot plot of the CUZD1 ELISA assay results.

FIG. 6 shows the receiver operating characteristic curve of benign versus pancreatic cancer stages I and II Multivariate Model of CA19-9 XR, CA-125 II and CUZD1 together.

FIG. 7 shows the receiver operating characteristic curve of benign versus pancreatic cancer stages I and II Multivariate Model of CA19-9 XR and CUZD1 together.

FIG. 8 shows the receiver operating characteristic curve of benign versus pancreatic cancer stages I and II Multivariate Model of CA19-9 XR and CA-125 II together.

FIG. 9 shows the receiver operating characteristic curve of benign versus pancreatic cancer stages I and II Multivariate Model of CA-125 II and CUZD1 together.

### DETAILED DESCRIPTION

The present invention discloses a panel of biomarkers that show a high sensitivity and specificity for the early detection of pancreatic cancer. The present invention is directed to analyzing or quantifying the levels of carbohydrate antigen 19-9 (CA19-9), carbohydrate antigen 125 (CA125) and zona pellucida-like domain-containing protein 1 (CUZD1) to identify, diagnose and treat pancreatic cancer in high risk individuals and patients in need thereof. The methods can be adapted for use in an automated system or a semi-automated system.

The methods described herein differ over previous pancreatic cancer diagnostic methods by using the unique combination of CA19-9, CA125, and CUZD1 markers to distinguish patients suffering from pancreatic cancer with healthy patients or those with benign pancreatic disease. Previous assays that use only one or two markers, such as CA19-9, CA19-9 and CUZD1, CA125 and CUZD1, etc., to classify patients who are healthy or who have benign pancreatic disease, but have difficulty classifying patients with early stage cancers. The disclosed combination of markers performed better than methods employing just one or two markers in differentiating patients with pancreatic cancer from healthy patients or patients with benign pancreatic disease. In addition, the following combination of markers can be measured to decipher subjects with early stage pancreatic cancer or late state pancreatic cancer, by quantifying the levels of this combination of markers.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The “area under curve” or “AUC” refers to area under a ROC curve. AUC under a ROC curve is a measure of accuracy. An area of 1 represents a perfect test, whereas an area of 0.5 represents an insignificant test. A preferred AUC may be at least approximately 0.700, at least approximately 0.750, at least approximately 0.800, at least approximately 0.850, at least approximately 0.900, at least approximately 0.910, at least approximately 0.920, at least approximately 0.930, at least approximately 0.940, at least approximately 0.950, at least approximately 0.960, at least approximately 0.970, at least approximately 0.980, at least approximately 0.990, or at least approximately 0.995.

“Benign pancreatic disease”, “benign”, and “pancreatic disease” as used herein interchangeably refer to pancreatic disease which is not cancer or has become cancer. Benign pancreatic disease includes pancreatitis, various types of cysts and tumors, pancreatic intraepithelial neoplasia (PanIN) and intraductal papillary mucinous neoplasm (IPMN) lesions, and mucinous cystic neoplasm (MCN).

The “confidence interval” or “CI” as used herein refers to an interval estimate of a population parameter used to indicate the reliability of an estimate. The confidence interval refers to the region containing the limits or band of a parameter with an associated confidence level that the bounds are large enough to contain the true parameter value. The bands may be single-sided to describe an upper or lower limit or double sided to describe both upper and lower limits. The region gives a range of values, bounded below by a lower confidence limit and from above by an upper confidence limit, such that one can be confident (at a pre-specified level such as 95% or 99%) that the true population parameter value is included within the confidence interval. Confidence intervals may be formed for any of the parameters used to describe the characteristic of interest. Confidence intervals may be used to estimate the population parameters from the sample statistics and allow a probabilistic quantification of the strength of the best estimate. A preferred confidence interval may be at least 90%, at least 91%, at least 92%, at least 93%, at least 94%, at least 95%, at least 96%, at least 97%, at least 98%, or at least 99%.

“Early stage pancreatic cancer” and “early stage” as used herein interchangeably refer to pancreatic cancer which is limited to the pancreas, outside the pancreas or nearby lymph nodes, but has not expanded into nearby major blood vessels or nerves or distant organs. Early stage pancreatic cancer includes stage 0, stage I and stage II pancreatic cancers. See Yachida, S., et al., 2010, Nature, 4 6 7, 114-419; see also National Comprehensive Cancer Network (NCCN) Guidelines Version 2.2012 Pancreatic Adenocarcinoma available at www.cancer.org/cancer/pancreaticcancer/detailedguide/pancreatic-cancer-staging.

“High risk individual” and “high risk patient” as used herein interchangeably refer to patients who have benign pancreatic disease or who have a family history of pancreatic history. A “family history of pancreatic history” or “familial pancreatic cancer” refers to an individual who has two or more first-degree relatives (parents, brothers, sisters, children) diagnosed with pancreatic cancer, three or more close relatives (first-degree relatives, grandparents, aunts, uncles, nieces, nephews, grandchildren, cousins) diagnosed with pancreatic cancer, or one relative diagnosed with pancreatic cancer before the age of 50. A high risk individual may also include subjects who have been diagnosed as having genes which are correlated with increased risk of pancreatic cancer. Genes that have been correlated with increased risk of pancreatic cancer include STK11/LKB1, PALB2, CDKN2A, BRCA1, BRCA2, PRSS1 and SPINK1.

“Interquartile range” or “IQR” as used herein means a measure of statistical dispersion, being equal to the difference between the upper and lower quartiles, IQR=Q3−Q1.

“Late stage pancreatic cancer” and “late stage” as used herein interchangeably refer to pancreatic cancer which has expanded into nearby major blood vessels, nerves or distant organs. Late stage pancreatic cancer includes stage III or stage IV pancreatic cancer.

“Normal” and “healthy” as used herein interchangeably refer to patients that do not have pancreatic cancer or pancreatic disease.

“Pancreatic cancer” as used herein refers to cancer that originates in pancreas. Pancreatic cancer includes exocrine pancreatic cancer, such as pancreatic adenocarcinoma, acinar cell carcinoma, adenocarcinoma, adenosquamous carcinoma, giant cell tumor, mucinous cystadenocarcinoma, pancreatoblastoma, serous cystadenocarcinoma, solid and pseudopapillary tumors, and papillar cystic neoplasms, endocrine pancreatic cancer, such as pancreatic neuroendocrine tumors (NET), islet cell tumors, islet cell carcinoma, pancreatic carcinoid, pancreatic endocrine tumor (PET), gastrinoma (Zollinger-Ellison Syndrome), glucagonoma, insulinoma, nonfunctional islet cell tumor, somatostatinoma, and vasoactive intestinal peptide-releasing tumor (VIPoma or Verner-Morrison Syndrome), and lymphoma of the pancreas. Adenocarcinoma may develop from a benign intraductal papillary-mucinous neoplasm (IPMN).

“Predetermined cutoff” and “predetermined level” as used herein refer to an assay cutoff value that is used to assess diagnostic, prognostic, or therapeutic efficacy results by comparing the assay results against the predetermined cutoff/level, where the predetermined cutoff/level already has been linked or associated with various clinical parameters (e.g., presence of disease, stage of disease, severity of disease, progression, non-progression, or improvement of disease, etc.). The disclosure provides exemplary predetermined levels. However, it is well-known that cutoff values may vary depending on the nature of the immunoassay (e.g., antibodies employed, reaction conditions, sample purity, etc.). It further is well within the ordinary skill of one in the art to adapt the disclosure herein for other immunoassays to obtain immunoassay-specific cutoff values for those other immunoassays based on the description provided by this disclosure. Whereas the precise value of the predetermined cutoff/level may vary between assays, the correlations as described herein should be generally applicable.

“Risk assessment,” “risk classification,” “risk identification,” or “risk stratification” of subjects (e.g., patients) as used herein refers to the evaluation of factors including biomarkers, to predict the risk of occurrence of future events including disease onset or disease progression, so that treatment decisions regarding the subject may be made on a more informed basis.

“Sample,” “biological sample”, “test sample,” “specimen,” “sample from a subject,” and “patient sample” as used herein may be used interchangeable and may be a sample of blood, tissue, urine, serum, plasma, amniotic fluid, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

Any cell type, tissue, or bodily fluid may be utilized to obtain a sample. Such cell types, tissues, and fluid may include sections of tissues such as biopsy and autopsy samples, frozen sections taken for histologic purposes, blood (such as whole blood), plasma, serum, sputum, stool, tears, mucus, saliva, bronchoalveolar lavage (BAL) fluid, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary.

“Stage 0 pancreatic cancer” as used herein refers to pancreatic cancer limited to a single layer of cells in the pancreas. The pancreatic cancer is not visible on imaging tests or to the naked eye. The tumor is confined to the top layers of pancreatic duct cells and has not invaded deeper tissues. It has not spread outside of the pancreas. Stage 0 tumors are sometimes referred to as pancreatic carcinoma in situ or pancreatic intraepithelial neoplasia III (PanIn III).

“Stage I pancreatic cancer” and “stage I” as used herein interchangeably refer to cancer confined or limited to the pancreas and has not spread to nearby lymph nodes. “Stage IA” refers to a tumor confined to the pancreas and is less than 2 cm in size. “Stage IB” refers to a tumor confined to the pancreas and is greater than 2 cm in size.

“Stage II pancreatic cancer” and “stage II” as used herein interchangeably refer to local spread cancer that has grown outside the pancreas or has spread to nearby lymph nodes. “Stage IIA refers to a tumor growing outside the pancreas but not into large blood vessels, nearby lymph nodes or distant sites. “Stage JIB” refers to a tumor either confined to the pancreas or growing outside the pancreas but has not spread into nearby large blood vessels or major nerves. Stage IIB may spread to nearby lymph nodes but has not spread to distant sites.

“Stage III pancreatic cancer” and “stage III” as used herein interchangeably refer to wider spread cancer that has expanded into nearby major blood vessels or nerves but has not metastasized. The tumor is growing outside the pancreas into nearby large blood vessels or major nerves and may or may not have spread to nearby lymph nodes. It has not spread to distant sites.

“Stage IV pancreatic cancer” and “stage IV” as used herein interchangeably refer to confirmed spread cancer that has spread to distant organs or sites. Stage IVA pancreatic cancer is locally confined, but involves adjacent organs or blood vessels, thereby hindering surgical removal. Stage IVA pancreatic cancer is also referred to as localized or locally advanced. Stage IVB pancreatic cancer has spread to distant organs, most commonly the liver. Stage IVB pancreatic cancer is also called metastatic

“Subject” and “patient” as used herein interchangeably refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. The subject or patient may be undergoing other forms of treatment.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. METHOD OF DIAGNOSING, PROGNOSTICATING, AND/OR STRATIFYING RISK OF PANCREATIC CANCER USING A COMBINATION OF MARKERS

The present invention is directed to methods of diagnosing, prognosticating, or stratifying risk of pancreatic cancer in a patient from whom the test or biological sample was obtained. The methods can be adapted for use in an automated system or a semi-automated system. By measuring the levels of CA19-9, CA125, and CUZD1, the method allows the early detection of pancreatic cancer to be more accurately diagnosed and subsequently treated more successfully, compared to other commercially available assays.

Generally, predetermined levels can be employed as a benchmark against which to assess results obtained upon assaying a test sample for CA19-9, CA125, and CUZD1. Generally, in making such a comparison, the predetermined levels are obtained by running a particular assay a sufficient number of times and under appropriate conditions such that a linkage or association of the analyte present, amount or concentration with a particular stage or endpoint of the pancreatic cancer with particular indicia can be made. Typically, the predetermined levels are obtained with assays of reference subjects (or populations of subjects). The CA19-9, CA125, and CUZD1 measured can include CA19-9, CA125, and CUZD1 fragments thereof, degradation products thereof, and/or enzymatic cleavage products thereof.

In particular, with respect to predetermined levels as employed for monitoring disease progression and/or treatment, the amount or concentration of CA19-9, CA125, and CUZD1 or CA19-9, CA125, and CUZD1 fragments may be “unchanged,” “favorable” (or “favorably altered”), or “unfavorable” (or “unfavorably altered”). “Elevated” or “increased” refers to an amount or a concentration in a test sample that is higher or greater than a typical or normal level or range (e.g., predetermined level), or is higher or greater than another reference level or range (e.g., earlier or baseline sample). The term “lowered” or “reduced” refers to an amount or a concentration in a test sample that is lower or less than a typical or normal level or range (e.g., predetermined level), or is lower or less than another reference level or range (e.g., earlier or baseline sample). The term “altered” refers to an amount or a concentration in a sample that is altered (increased or decreased) over a typical or normal level or range (e.g., predetermined level), or over another reference level or range (e.g., earlier or baseline sample).

The typical or normal levels or ranges for CA19-9, CA125, and CUZD1 are defined in accordance with standard practice. A so-called altered level or alteration can be considered to have occurred when there is any net change as compared to the typical or normal level or range, or reference level or range that cannot be explained by experimental error or sample variation. Thus, the level measured in a particular sample will be compared with the level or range of levels determined in similar samples from a so-called normal subject. In this context, a “normal” (sometimes termed “control” or “healthy”) subject is an individual with no detectable pancreatic disease or cancer, and a “normal” patient or population is/are one(s) that exhibit(s) no detectable pancreatic disease or cancer, respectively, for example. An “apparently normal subject” is one in which CA19-9, CA125, and CUZD1 has not been or is being assessed. The level of an analyte is said to be “elevated” when the analyte is normally undetectable (e.g., the normal level is zero, or within a range of from about 25 to about 75 percentiles of normal populations), but is detected in a test sample, as well as when the analyte is present in the test sample at a higher than normal level.

Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having lung cancer. A description of ROC analysis as applied according to the present disclosure is provided in P. J. Heagerty et al., Time-dependent ROC curves for censored survival data and a diagnostric marker, Biometrics 56:337-44(2000), the disclosure of which is hereby incorporated by reference in its entirety. Alternatively, cutoff values can be determined by a quartile analysis of biological samples of a patient group. For example, a cutoff value can be determined by selecting a value that corresponds to any value in the 25th-75th percentile range, preferably a value that corresponds to the 25th percentile, the 50th percentile or the 75th percentile, and more preferably the 75th percentile.

Such statistical analyses can be performed using any method known in the art and can be implemented through any number of commercially available software packages (e.g., from Analyse-it Software Ltd., Leeds, UK; StataCorp LP, College Station, Tex.; SAS Institute Inc., Cary, N.C.).

Thus, inter alia, the disclosure provides a method of screening for a subject having, or at risk of having pancreatic cancer.

a. Methods of Providing a Diagnosis of a Subject Having Pancreatic Cancer

The method described herein can be used to provide a diagnosis of a subject having pancreatic cancer by determining the levels of CA19-9, CA125, and CUZD1 in a subject. The method showed that CA19-9, CA125, and CUZD1 concentrations were higher in patients with pancreatic cancer. The method includes the steps of (a) obtaining a biological sample from a subject, (b) determining the levels of CA19-9, CA125, and CUZD1 in the biological sample, (c) comparing the levels of CA19-9, CA125, and CUZD1 in the biological sample to reference levels of CA19-9, CA125, and CUZD1, (d) identifying the subject as having pancreatic cancer if the levels of CA19-9, CA125, and CUZD1 in the biological sample are greater than the reference levels of CA19-9, CA125, and CUZD1, and (e) administering a therapy against pancreatic cancer to the subject identified as having pancreatic cancer or a means by which to monitor pancreatic status in said subject identified as at risk of having pancreatic cancer.

The reference level in this method can be the levels of CA19-9, CA125, and CUZD1 in a patient having pancreatic cancer. Levels higher than or equal to 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, or 20 ng/mL of CA19-9 in serum in combination with levels higher than or equal to 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, or 25 ng/mL of CA125 in serum and levels higher than or equal to 1.0 ng/mL, 1.1 ng/mL, 1.2 ng/mL, 1.3 ng/mL, 1.4 ng/mL, 1.5 ng/mL, 1.6 ng/mL, 1.7 ng/mL, 1.8 ng/mL, 1.9 ng/mL, and 2.0 ng/mL of CUZD1 in serum identify the subject as having pancreatic cancer.

(1) Early Stage

The method described herein can be used to provide a diagnosis of a subject having early stage pancreatic cancer by determining the levels of CA19-9, CA125, and CUZD1 in a subject. The method showed that CA19-9, CA125, and CUZD1 concentrations were higher in patients with early stage pancreatic cancer compared to healthy patients. The method includes the steps of (a) obtaining a biological sample from a subject, (b) determining the levels of CA19-9, CA125, and CUZD1 in the biological sample, (c) comparing the levels of CA19-9, CA125, and CUZD1 in the biological sample to a reference levels of CA19-9, CA125, and CUZD1, (d) identifying the subject as having early stage pancreatic cancer if the levels of CA19-9, CA125, and CUZD1 in the biological sample are greater than the reference levels of CA19-9, CA125, and CUZD1, and (e) administering a therapy against pancreatic cancer to the subject identified as having early stage pancreatic cancer or a means by which to monitor pancreatic status in said subject identified as at risk of having early stage pancreatic cancer.

The reference level in this method can be the levels of CA19-9, CA125, and CUZD1 in a patient having early stage pancreatic cancer. Levels higher than or equal to 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, or 20 ng/mL of CA19-9 in serum in combination with levels higher than or equal to 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, or 25 ng/mL of CA125 in serum and levels higher than or equal to 1.0 ng/mL, 1.1 ng/mL, 1.2 ng/mL, 1.3 ng/mL, 1.4 ng/mL, 1.5 ng/mL, 1.6 ng/mL, 1.7 ng/mL, 1.8 ng/mL, 1.9 ng/mL, and 2.0 ng/mL of CUZD1 in serum identify the subject as having early stage pancreatic cancer.

(2) Stage I

The method described herein can be used to provide a diagnosis of a subject having stage I pancreatic cancer by determining the levels of CA19-9, CA125, and CUZD1 in a subject. The method showed that CA19-9, CA125, and CUZD1 concentrations were higher in patients with stage I pancreatic cancer compared to healthy patients. The method includes the steps of (a) obtaining a biological sample from a subject, (b) determining the levels of CA19-9, CA125, and CUZD1 in the biological sample, (c) comparing the levels of CA19-9, CA125, and CUZD1 in the biological sample to a reference levels of CA19-9, CA125, and CUZD1, (d) identifying the subject as having stage I pancreatic cancer if the levels of CA19-9, CA125, and CUZD1 in the biological sample is greater than the reference levels of CA19-9, CA125, and CUZD1, and (e) administering a therapy against stage I pancreatic cancer to the subject identified as having stage I pancreatic cancer or a means by which to monitor pancreatic status in said subject identified as at risk of having stage I pancreatic cancer.

The reference level in this method can be the levels of CA19-9, CA125, and CUZD1 in a patient having stage I pancreatic cancer. Levels higher than or equal to 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, or 20 ng/mL of CA19-9 in serum in combination with levels higher than or equal to 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, or 25 ng/mL of CA125 in serum and levels higher than or equal to 1.0 ng/mL, 1.1 ng/mL, 1.2 ng/mL, 1.3 ng/mL, 1.4 ng/mL, 1.5 ng/mL, 1.6 ng/mL, 1.7 ng/mL, 1.8 ng/mL, 1.9 ng/mL, and 2.0 ng/mL of CUZD1 in serum identify the subject as having stage I pancreatic cancer.

(3) Late Stage

The method described herein can be used to provide a diagnosis of a subject having late stage pancreatic cancer by determining the levels of CA19-9, CA125, and CUZD1 in a subject. The method showed that CA19-9, CA125, and CUZD1 concentrations were higher in patients with late stage pancreatic cancer compared to healthy patients. The method includes the steps of (a) obtaining a biological sample from a subject, (b) determining the levels of CA19-9, CA125, and CUZD1 in the biological sample, (c) comparing the levels of CA19-9, CA125, and CUZD1 in the biological sample to a reference levels of CA19-9, CA125, and CUZD1, (d) identifying the subject as having late stage pancreatic cancer if the levels of CA19-9, CA125, and CUZD1 in the biological sample are greater than the reference levels of CA19-9, CA125, and CUZD1, and (e) administering a therapy against pancreatic cancer to the subject identified as having late stage pancreatic cancer or a means by which to monitor pancreatic status in said subject identified as at risk of having late stage pancreatic cancer.

The reference level in this method can be the levels of CA19-9, CA125, and CUZD1 in a patient having late stage pancreatic cancer. Levels higher than or equal to 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, or 20 ng/mL of CA19-9 in serum in combination with levels higher than or equal to 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, or 25 ng/mL of CA125 in serum and levels higher than or equal to 1.0 ng/mL, 1.1 ng/mL, 1.2 ng/mL, 1.3 ng/mL, 1.4 ng/mL, 1.5 ng/mL, 1.6 ng/mL, 1.7 ng/mL, 1.8 ng/mL, 1.9 ng/mL, and 2.0 ng/mL of CUZD1 in serum identify the subject as having late stage pancreatic cancer.

b. Methods for Determining the Risk of a Subject of Developing Pancreatic Cancer

The methods described herein also can be used to determine whether or not a subject has or is at risk of developing pancreatic cancer by determining the levels of CA19-9, CA125, and CUZD1 in a subject. Thus, in particular embodiments, the disclosure also provides a method for determining whether a subject having, or at risk for, pancreatic cancer, discussed herein and known in the art, is a candidate for treatment or monitoring. Generally, the subject is a high risk individual, i.e., a patient who has a family history of pancreatic history, who has been diagnosed as having benign pancreatic disease, who has actually been diagnosed as having or being at risk for, pancreatic cancer, and/or who demonstrates unfavorable concentrations or amounts of CA19-9, CA125, and CUZD1, as described herein.

Specifically, such a method can comprise the steps of: (a) determining the concentrations or amounts in a test sample from a subject of CA19-9, CA125, and CUZD1 using methods known in the art); and (b) comparing the concentrations or amounts CA19-9, CA125, and CUZD1 determined in step (a) with predetermined levels, wherein, if the concentrations or amounts of CA19-9, CA125, and CUZD1 determined in step (a) is favorable with respect to predetermined levels, then the subject is determined not to have or be at risk for pancreatic cancer as discussed herein and known in the art. However, if the concentrations or amounts of CA19-9, CA125, and CUZD1 determined in step (a) are unfavorable with respect to the predetermined levels, then the subject is determined to have or be at risk for pancreatic cancer as discussed herein and known in the art. The pancreatic cancer may be early or late stage pancreatic cancer.

c. Methods of Determining if a Subject is Suffering from Pancreatic Cancer

The subject identified in the methods described above having levels of CA19-9, CA125, and CUZD1 over the values discussed above are identified as patients suffering from pancreatic cancer. The subjects are then treated for pancreatic cancer. Moreover, the methods described herein also can be used to determine whether a subject predisposed to or suffering from pancreatic cancer and will benefit from treatment or periodic monitoring. The method of “monitoring the treatment of pancreatic cancer in a subject” as described herein further optimally also can encompass selecting or identifying candidates for pancreatic cancer treatments, such as surgery, radiation therapy, targeted therapy, and chemotherapy.

d. Methods of Monitoring the Progression of Pancreatic Cancer in a Subject

The methods described herein also can be used to monitor the progression of pancreatic cancer in a subject by determining the levels of CA19-9, CA125, and CUZD1 in a subject. Optimally, the method includes the steps of (a) determining the concentrations or amounts of CA19-9, CA125, and CUZD1 in a test sample from a subject, (b) determining the concentrations or amounts of CA19-9, CA125, and CUZD1 in a later test sample from a subject, and (c) comparing the concentrations or amounts of CA19-9, CA125, and CUZD1 as determined in step (b) with the concentrations or amounts of CA19-9, CA125, and CUZD1 determined in step (a), wherein if the concentrations or amounts determined in step (b) is unchanged or is unfavorable when compared to the concentrations or amounts of CA19-9, CA125, and CUZD1 determined in step (a), then the disease in the subject is determined to have continued, progressed or worsened. By comparison, if the concentrations or amounts of CA19-9, CA125, and CUZD1 as determined in step (b) is favorable when compared to the concentrations or amounts of CA19-9, CA125, and CUZD1 as determined in step (a), then the pancreatic cancer in the subject is determined to have discontinued, regressed or improved.

Optionally, the method further comprises comparing the concentrations or amounts of CA19-9, CA125, and CUZD1 as determined in step (b), for example, with predetermined levels. Further, optionally the method comprises treating the subject with one or more of surgery, chemotherapy, radiation therapy, targeted therapy, biological therapy, palliative care and combinations thereof (collectively, “treatment regimens”) for a period of time if the comparison shows that the concentrations or amounts of CA19-9, CA125, and CUZD1 as determined in step (b), for example, are unfavorably altered with respect to the predetermined levels.

Still further, the methods can be used to monitor treatment in a subject receiving treatment with one or more treatment regimens. Specifically, such methods involve providing a first test sample from a subject before the subject has been administered one or more treatment regimens. Next, the concentrations or amounts in a first test sample from a subject of CA19-9, CA125, and CUZD1 are determined (e.g., using methods known in the art). After the concentrations or amounts of CA19-9, CA125, and CUZD1 are determined, optionally the concentrations or amounts of CA19-9, CA125, and CUZD1 are then compared with predetermined levels. If the concentrations or amounts of CA19-9, CA125, and CUZD1 as determined in the first test sample are lower than the predetermined levels, then the subject is not treated with one or more treatment regimens or alternatively, the subject may be treated with one or more treatment regimens. If the concentrations or amounts of CA19-9, CA125, and CUZD1 as determined in the first test sample are higher than the predetermined levels, then the subject is treated with one or more treatment regimens for a period of time or alternatively, the subject is not treated with one or more treatment regimens. The period of time that the subject is treated with the one or more treatment regimens can be determined by one skilled in the art (for example, the period of time can be from about seven (7) days to about two years, preferably from about fourteen (14) days to about one (1) year).

During the course of treatment with the one or more treatment regimens, second and subsequent test samples are then obtained from the subject. The number of test samples and the time in which said test samples are obtained from the subject are not critical. For example, a second test sample could be obtained seven (7) days after the subject is first administered the one or more treatment regimens, a third test sample could be obtained two (2) weeks after the subject is first administered the one or more treatment regimens, a fourth test sample could be obtained three (3) weeks after the subject is first administered the one or more treatment regimens, a fifth test sample could be obtained four (4) weeks after the subject is first administered the one or more treatment regimens, etc.

After each second or subsequent test sample is obtained from the subject, the concentrations or amounts CA19-9, CA125, and CUZD1 are determined in the second or subsequent test sample (e.g., using methods known in the art). The concentrations or amounts of CA19-9, CA125, and CUZD1 as determined in each of the second and subsequent test samples are then compared with the concentrations or amounts of CA19-9, CA125, and CUZD1 as determined in the first test sample (e.g., the test sample that was originally optionally compared to the predetermined level). If the concentrations or amounts of CA19-9, CA125, and CUZD1 as determined in step (c) are favorable when compared to the concentrations or amounts of CA19-9, CA125, and CUZD1 as determined in step (a), then the disease in the subject is determined to have discontinued, regressed, or improved, and the subject should continue to be administered the one or treatment regimens of step (b). However, if the concentrations or amounts determined in step (c) are unchanged or are unfavorable when compared to the concentrations or amounts of CA19-9, CA125, and CUZD1 as determined in step (a), then the disease in the subject is determined to have continued, progressed or worsened, and the subject should be treated with a higher concentration of the one or more treatment regimens administered to the subject in step (b) or the subject should be treated with one or more treatment regimens that are different from the one or more treatment regimens administered to the subject in step (b). Specifically, the subject can be treated with one or more treatment regimens that are different from the one or more treatment regimens that the subject had previously received to decrease or lower said subject's CA19-9, CA125, and CUZD1 levels.

Furthermore, the above assays can be performed using a first test sample obtained from a subject where the first test sample is obtained from one source, such as urine, serum, or plasma. Optionally the above assays can then be repeated using a second test sample obtained from the subject where the second test sample is obtained from another source. For example, if the first test sample was obtained from urine, the second test sample can be obtained from serum or plasma. The results obtained from the assays using the first test sample and the second test sample can be compared. The comparison can be used to assess the status of a disease or condition in the subject

e. Methods of Determining the Stage of Pancreatic Cancer in a Subject

The present invention is directed to a method for distinguishing a subject suffering from early stage pancreatic cancer from late stage pancreatic cancer by quantifying the levels of CA19-9, CA125, and CUZD1. The method includes the steps of (1) obtaining a biological sample from a subject, (2) determining the level of CA19-9, CA125, and CUZD1 in the biological sample, (3) comparing the levels of CA19-9, CA125, and CUZD1 in the biological sample to first reference levels of CA19-9, CA125, and CUZD1 and second reference levels of CA19-9, CA125, and CUZD1; and (4) correlating the levels of CA19-9, CA125, and CUZD1 in the biological sample with the stage of pancreatic cancer in the subject, wherein if the levels of CA19-9, CA125, and CUZD1 are greater than the first reference levels of CA19-9, CA125, and CUZD1 but less than the second reference levels of CA19-9, CA125, and CUZD1, the subject is determined to have early stage pancreatic cancer, and wherein if the levels of CA19-9, CA125, and CUZD1 are greater than the first reference levels of CA19-9, CA125, and CUZD1 and the second reference levels of CA19-9, CA125, and CUZD1, the subject is determined to have late stage pancreatic cancer. The subject may then be administered a therapy against early stage or late stage pancreatic cancer to the subject identified as having early stage or late stage pancreatic cancer or a means by which to monitor pancreatic status in said subject identified as at risk of having pancreatic cancer.

### 3. BIOMARKERS (CONTROLS)

The method as described above may further comprise measuring an additional biomarker in combination with the specific combinations discussed above to determine whether a subject is suffering from pancreatic cancer. The method may further comprise determining the level of at least one additional biomarker of pancreatic cancer in the biological sample and comparing the level of the at least one additional biomarker of pancreatic cancer to a reference concentration value for the at least one biomarker of pancreatic cancer. The additional biomarker may be CEA, HE4, NGAL, TIMP-1, CA15-3, AGR2, LRG1, COL6A3, REG1B and SYCN. For example, the method may further comprise determining the level of CEA in the biological sample and comparing the level of CEA in the biological sample to a reference level of, wherein levels of CA19-9, CA125, CUZD1, and CEA in the biological sample greater than the reference levels of CA19-9, CA125, CUZD1, and CEA identifies the subject as having pancreatic cancer.

(1) CA19-9

Carbohydrate antigen (CA) 19-9 (also referred to herein as “CA 19-9 XR” and “CA 19-9”) may be used in the methods described above in combination with CA125 and CUZD1. CA19-9, also known as cancer antigen 19-9, Sialylated Lewis (a) antigen, gastrointestinal cancer antigen, cancer antigen-GI, and CA-GI, is encoded by CA19-9 and is associated with digestive tract cancer and found in many adenocarcinomas of the digestive tract, especially pancreatic tumors. CA19-9 has been FDA approved for use as an aid in the management of pancreatic cancer and intended to be used in conjunction with other diagnostic information such as CT and MRI imaging procedures. CA19-9 belongs to the sialylated Lewis blood group antigen. Some individuals may be undetectable for CA19-9 because they are Lewis antigen negative. Increased serum CA19-9 may also be elevated in patients with nonmalignant conditions such as pancreatitis and other gastrointestinal disorders. CA19-9 alone was insufficient at detecting early pancreatic cancer, therefore, adding biomarkers to CA19-9 in a panel improved its sensitivity and specificity performance for early detection of pancreatic cancer.

(2) CA125

Carbohydrate antigen (CA) 125 (also referred to herein as “CA 125 II” and “CA 125”) may be used in the methods described above in combination with CA19-9 and CUZD1. CA125, also known as CA-125, cancer antigen 125, carbohydrate antigen 125, Mucin 16, and MUC16, is encoded by MUC16 and is most commonly seen in tumors of the ovary and occasionally in breast, kidney, and gastrointestinal tract tumors and normal tissues. CA125 is tumor-associated but not tumor-specific. CA125 is FDA approved for monitoring response to therapy for patients with epithelial ovarian cancer and is useful for monitoring the course of disease in patients with invasive ovarian cancer. Values of CA125 are defined by using the OC 125 monoclonal antibody.

(3) CUZD1

CUB and zona pellucida-like domains 1 (CUZD1) may be used in the methods described above in combination with CA19-9 and CA125. CUZD1, also known as CUB and ZP domain-containing protein 1, estrogen regulated gene 1 (ERG-1), transmembrane protein UO-44, and UO44, is encoded by the CUZD1 gene and may play a role in the uterus during late pregnancy and/or trypsin activation in pancreatic acinar cells. CUZD1 antiserum inhibits cell attachment and proliferation of ovarian cancer cells and may be involved in these processes.

(4) Others Markers

(a) CEA

Carcinoembryonic Antigen (CEA) is a tumor associated antigen characterized by a glycoprotein that is approximately 200 kDa in size. CEA is FDA approved and intended to be used as an aid in the prognosis and management of cancer patients in patients with changing concentrations of CEA. Clinical relevance has been shown in colorectal, gastric, lung, prostate, pancreatic, and ovarian cancers.

(b) HE4

Human epididymis protein 4 (HE4) is part of the family of whey acidic four-disulfide core (WFDC) proteins with trypsin inhibitor properties. Other members of the family include SLPI, Elafin, and PS20 (WFDC1). The HE4 gene encodes a 13 kDa protein, however, the mature glycosylated form is approximately 20-25 kDa. HE4 is FDA approved for the aid in monitoring recurrence or progressive disease in patients with epithelial ovarian cancer.

(c) NGAL

Neutrophil gelatinase-associated lipocalin (NGAL) is FDA approved for use in urine as an indication of kidney injury and used to aid in the diagnosis of the disease. Studies have shown that urinary NGAL is an early marker of acute kidney injury (AKI). The use of NGAL in serum is not FDA approved and is experimental for research use only. NGAL is one of the earliest proteins that is induced in the kidney after kidney injury and is seen as little as 2 hours after the injury.

(d) AGR2

Anterior gradient homolog 2 (AGR2), also known as hAG-2 and Gob-4, is the human orthologue of the secreted Xenopus laevis anterior gradient protein, XAG-2. The expression of XAG-2 induces cement gland differentiation and expression of neural marker genes in a fibroblast growth factor-dependent manner. Up-regulation of the AGR2 gene has been reported in ovarian, breast cancer, prostate cancer endometrial cancer and renal cancer.

(e) LRG1

Using proteomic analysis, the biomarker leucine-rich a-2-glycoprotein (LRG1) was shown to be up-regulated in biliary tract cancer, and was validated using a serum ELISA assay

(f) TIMP-1

Tissue inhibitor of metalloproteinase-1 (TIMP-1) is a glycoprotein of about 28.5 kDa and has a broad array of inhibitory properties toward all the known 22 matrix metalloproteinases (MMPs). TIMP-1 binds with high affinity to the active sites of the MMPs to form complexes. TIMP-1 elevation has been shown to be potentially useful in colorectal cancer, liver fibrosis, cardiac mortality and myocardial infarction.

(g) CA 15-3

Carbohydrate antigen 15-3 (CA 15-3) assay values are defined by using the 115D8 and DF3 monoclonal antibodies (ARCHITECT® CA15-3). Monoclonal antibody 115D8 was raised against human milk-fat globule membranes, and monoclonal antibody DF3 was raised against a membrane enriched fraction of metastatic human breast carcinoma. These react with epitopes expressed by a family of high molecular weight glycoproteins designated as polymorphic epithelial mucins (PEMs). CA15-3 is intended to be used as an aid in the management of Stage II and III breast cancer.

(h) REG1b

REG1b is a type I subclass member of the Reg gene family, which is a family grouped into four subclasses of genes, types I, II, III and IV based on the primary structures of the encoded proteins. This gene encodes a protein secreted by the exocrine pancreas that is highly similar to the REG1A protein, which is associated with islet cell regeneration. REG1b may act as an inhibitor of spontaneous calcium carbonate precipitation and, may be associated with neuronal sprouting in brain, and with brain and pancreas regeneration.

(i) COL6A3

COL6A3 acts as a cell-binding protein. This gene encodes the alpha-3 chain, one of the three alpha chains of type VI collagen, a beaded filament collagen found in most connective tissues, type A domains, which are found in the amino terminal globular domain of all the alpha chains. These domains have been shown to bind extracellular matrix proteins, an interaction that explains the importance of this collagen in organizing matrix components. Mutations in this gene are also a cause of Ullrich congenital muscular dystrophy.

(j) SYNC

Syncoilin (SYNC, also referred herein as “SYCN”) is an intermediate filament protein related to desmin-related myopathy, muscular dystrophies and neuromuscular diseases. SYNC protein is highly expressed in skeletal and cardiac muscle. The protein links the dystrophin associated protein complex to desmin filaments in muscle and may have a structural role in striated muscle.

### 4. MEANS FOR CONFIRMING PANCREATIC STATUS IN A SUBJECT

The subject identified in the methods described above having levels of CA19-9, CA125, and CUZD1 greater than or equal to the values discussed above is identified as having pancreatic cancer. The subject may then be administered a means for confirming the pancreatic status. A means for confirming the pancreatic status may include performing biopsy, such as during endoscopy or with an operation, such as fine needle, percutaneous needle biopsy, or EUS guided, imaging with ultrasound, MRI, PET scan, CT scan, ultrasound, endoscopic ultrasound (EUS), endoscopic retrograde cholangiopancreatography (ERCP), EUS cholangiopancreatography, MRI/magnetic resonance cholangiopancreatography (MRI/MRCP) or laparoscopy, measuring pancreatic function, such as a chromogranin A test, a somatostatin receptor scintigraphy, an angiogram, blood chemistry tests, laparotomy, intraoperative ultrasound, a bone scan, fasting serum somatostatin test, stool analysis, serum VIP test, fasting serum glucagon test, fasting serum glucose and insulin test, and determining CA19-9, CA125, CUZD1 levels and/or other biomarkers, alone or in combination, at periodic intervals.

### 5. TREATMENT OF SUBJECTS SUFFERING FROM PANCREATIC CANCER

The subject identified in the methods described above having levels of CA19-9, CA125, and CUZD1 greater than or equal to the values discussed above is identified as a patient suffering from pancreatic cancer. The subject is then treated for the pancreatic cancer. Treatment of pancreatic cancer may include alone or in combination surgery, chemotherapy, radiation therapy, targeted therapy, biological therapy, and palliative care. Chemotherapy and radiation may be administered together, prior to (neoadjuvant therapy), after, or without surgery, to slow pancreatic cancer's growth. Chemotherapy or radiation therapy or both can also be used in conjunction with surgery for resectable and unresectable pancreatic cancer in order to shrink pancreatic cancer before surgery, improving the chances of resection (neoadjuvant therapy) and/or prevent or delay pancreatic cancer from returning after surgery (adjuvant therapy). Palliative care may be administered to reduce discomfort for people whose pancreatic cancer cannot be cured.

Surgery may include total pancreatectomy, distal pancreatectomy, and Whipple procedure, also known as pancreaticoduodenectomy, pancreatoduodenectomy, and Kausch-Whipple. Chemotherapy may include 5-fluorouracil (5-FU), capecitabine (Xeloda®), gemcitabine (Gemzar), FOLFIRINOX (5-FU/leucovorin/oxaliplatin/irinotecan), erlotinib, cisplatin (Platinol®), Abraxane® (nab-paclitaxel), Irinotecan (Camptosar®), Alimta® (pemetrexed), Eloxatin® (oxaliplatin); Ellence® (epirubicin), (doxorubicin), Tarceva® (erlotinib), Taxotere® (docetaxel), or combinations thereof. Chemotherapy may also include drugs targeting Ras. Radiation therapy may include X-rays, gamma rays, charged particles (proton beam radiation therapy), external-beam radiation therapy, internal radiation therapy (brachytherapy), systemic radiation therapy, three-dimensional conformal radiation therapy (3D-CRT), Intensity-Modulated Radiation Therapy (IMRT), CyberKnife®, and photodynamic therapy, which utilizes a photosensitizing agent and light. Radiation therapy may be administered during a series of daily treatments, usually over a period of weeks. Targeted therapy may include Herceptin® (trastuzumab) and Erbitux® (cetuximab). Biological therapy may include immunotherapy to boost a person's immune system to fight disease, such as vaccines, and Virulizin®.

a. Early Stage Pancreatic Cancer—Stage I and Stage II

The subject identified in the methods described above having levels of CA19-9, CA125, and CUZD1 greater than or equal to the values discussed above is identified as a patient suffering from early stage pancreatic cancer. The subject is then treated for the early stage pancreatic cancer. Treatment of early stage pancreatic cancer may include surgery, chemotherapy, radiation therapy, targeted therapy, biological therapy, palliative care, or combinations thereof, as described above.

(1) Stage I

The subject identified in the methods described above having levels of CA19-9, CA125, and CUZD1 greater than or equal to the values discussed above is identified as a patient suffering from stage I pancreatic cancer. The subject is then treated for stage I pancreatic cancer. Treatment of stage I pancreatic cancer may include surgery, chemotherapy, radiation therapy, targeted therapy, biological therapy, palliative care, or combinations thereof, as described above.

b. Late Stage Pancreatic Cancer—Stage III and Stage IV

The subject identified in the methods described above having levels of CA19-9, CA125, and CUZD1 greater than or equal to the values discussed above is identified as a patient suffering from late stage pancreatic cancer. The subject is then treated for the late stage pancreatic cancer. Treatment of late stage pancreatic cancer may include surgery, chemotherapy, radiation therapy, targeted therapy, biological therapy, palliative care, or combinations thereof, as described above.

### 6. MEANS FOR MONITORING PANCREATIC STATUS

The subject identified in the methods described above having levels of CA19-9, CA125, and CUZD1 greater than or equal to the values discussed above is identified as a patient at risk of having pancreatic cancer. The subject is then administered a means for monitoring the pancreatic status. A means for confirming the pancreatic status may include performing biopsy, such as during endoscopy or with an operation, such as fine needle, percutaneous needle biopsy, or EUS guided, imaging with ultrasound, MRI, PET scan, CT scan, ultrasound, endoscopic ultrasound (EUS), endoscopic retrograde cholangiopancreatography (ERCP), EUS cholangiopancreatography, MRI/magnetic resonance cholangiopancreatography (MRI/MRCP) or laparoscopy, measuring pancreatic function, such as a chromogranin A test, a somatostatin receptor scintigraphy, an angiogram, blood chemistry tests, laparotomy, intraoperative ultrasound, a bone scan, fasting serum somatostatin test, stool analysis, serum VIP test, fasting serum glucagon test, fasting serum glucose and insulin test, and determining CA19-9, CA125, CUZD1 levels and/or other biomarkers, alone or in combination, at periodic intervals.

### 7. IMMUNOASSAYS TO MEASURE MARKERS

The methods described above quantify levels of the markers, CA19-9, CA125, and CUZD1. The methods may determine the levels of CA19-9, CA125, and CUZD1 using an immunological method with molecules binding to CA19-9, CA125, and CUZD1. Examples of antibodies that can be used include a polyclonal antibody, a monoclonal antibody, a human antibody, an immunoglobulin molecule, a disulfide linked Fv, a monoclonal antibody, an affinity matured, a scFv, a chimeric antibody, a single domain antibody, a CDR-grafted antibody, a diabody, a humanized antibody, a multispecific antibody, a Fab, a dual specific antibody, a DVD, a Fab′, a bispecific antibody, a F(ab′)2, a Fv and combinations thereof. The immunological method may include (a) measuring the levels of CA19-9 by: (i) contacting the test sample with at least one capture antibody, wherein the capture antibody binds to an epitope on CA19-9 or a fragment of CA19-9 to form a capture antibody-CA19-9 antigen complex; (ii) contacting the capture antibody-CA19-9 antigen complex with at least one detection antibody comprising a detectable label, wherein the detection antibody binds to an epitope on CA19-9 that is not bound by the capture antibody and forms a capture antibody-CA19-9 antigen-detection antibody complex; and (iii) determining the CA19-9 levels in the test sample based on the signal generated by the detectable label in the capture antibody-CA19-9 antigen-detection antibody complex formed in (a)(ii); (b) measuring the levels of CA125 by: (i) contacting the test sample with at least one capture antibody, wherein the capture antibody binds to an epitope on CA125 or a fragment of CA125 to form a capture antibody-CA125 antigen complex; (ii) contacting the capture antibody-CA125 antigen complex with at least one detection antibody comprising a detectable label, wherein the detection antibody binds to an epitope on CA125 that is not bound by the capture antibody and forms a capture antibody-CA125 antigen-detection antibody complex; and (iii) determining the CA125 levels in the test sample based on the signal generated by the detectable label in the capture antibody-CA125 antigen-detection antibody complex formed in (b)(ii); and (c) measuring the levels of CUZD1 by: (i) contacting the test sample with at least one capture antibody, wherein the capture antibody binds to an epitope on CUZD1 or a fragment of CUZD1 to form a capture antibody-CUZD1 antigen complex; (ii) contacting the capture antibody-CUZD1 antigen complex with at least one detection antibody comprising a detectable label, wherein the detection antibody binds to an epitope on CUZD1 that is not bound by the capture antibody and forms a capture antibody-CUZD1 antigen-detection antibody complex; and (iii) determining the CUZD1 levels in the test sample based on the signal generated by the detectable label in the capture antibody-CUZD1 antigen-detection antibody complex formed in (c)(ii).

The markers, i.e., CA19-9, CA125, and CUZD1, may be analyzed for the methods described above using an immunoassay. The presence or amount of marker can be determined using antibodies that specifically bind to the marker. Any immunoassay may be utilized. The immunoassay may be an enzyme-linked immunoassay (ELISA), radioimmunoassay (RIA), a competitive inhibition assay, such as forward or reverse competitive inhibition assays, a fluorescence polarization assay, or a competitive binding assay, for example. The ELISA may be a sandwich ELISA. Specific immunological binding of the antibody to the marker can be detected via direct labels, such as fluorescent or luminescent tags, metals and radionuclides attached to the antibody or via indirect labels, such as alkaline phosphatase or horseradish peroxidase.

The use of immobilized antibodies or fragments thereof may be incorporated into the immunoassay. The antibodies may be immobilized onto a variety of supports, such as magnetic or chromatographic matrix particles, the surface of an assay plate (such as microtiter wells), pieces of a solid substrate material, and the like. An assay strip can be prepared by coating the antibody or plurality of antibodies in an array on a solid support. This strip can then be dipped into the test biological sample and then processed quickly through washes and detection steps to generate a measurable signal, such as a colored spot.

The Sandwich ELISA measures the amount of antigen between two layers of antibodies (i.e. capture and a detection antibody). The marker, i.e., CA19-9, CA125, and CUZD1, to be measured may contain at least two antigenic sites capable of binding to antibody. Either monoclonal or polyclonal antibodies may be used as the capture and detection antibodies in the sandwich ELISA.

Generally, at least two antibodies are employed to separate and quantify the marker, i.e., CA19-9, CA125, or CUZD1, in a test sample. More specifically, the at least two antibodies bind to certain epitopes of the marker forming an immune complex which is referred to as a “sandwich”. One or more antibodies can be used to capture the marker in the test sample (these antibodies are frequently referred to as a “capture” antibody or “capture” antibodies) and one or more antibodies is used to bind a detectable (namely, quantifiable) label to the sandwich (these antibodies are frequently referred to as the “detection” antibody or “detection” antibodies). In a sandwich assay, both antibodies binding to their epitope may not be diminished by the binding of any other antibody in the assay to its respective epitope. In other words, antibodies may be selected so that the one or more first antibodies brought into contact with a test sample suspected of containing the marker do not bind to all or part of an epitope recognized by the second or subsequent antibodies, thereby interfering with the ability of the one or more second detection antibodies to bind to the marker.

In a preferred embodiment, a test sample suspected of containing the marker, i.e., CA19-9, CA125, or CUZD1, can be contacted with at least one first capture antibody (or antibodies) and at least one second detection antibodies either simultaneously or sequentially. In the sandwich assay format, a test sample suspected of containing the marker is first brought into contact with the at least one first capture antibody that specifically binds to a particular epitope under conditions which allow the formation of a first antibody-marker complex. If more than one capture antibody is used, a first multiple capture antibody-marker complex is formed. In a sandwich assay, the antibodies, preferably, the at least one capture antibody, are used in molar excess amounts of the maximum amount of marker expected in the test sample.

Optionally, prior to contacting the test sample with the at least one first capture antibody, the at least one first capture antibody can be bound to a solid support which facilitates the separation the first antibody-marker complex from the test sample. Any solid support known in the art can be used, including but not limited to, solid supports made out of polymeric materials in the forms of wells, tubes or beads. The antibody (or antibodies) can be bound to the solid support by adsorption, by covalent bonding using a chemical coupling agent or by other means known in the art, provided that such binding does not interfere with the ability of the antibody to bind the marker. Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl)carbodiimide.

After the test sample suspected of containing the marker is brought into contact with the at least one first capture antibody, the test sample is incubated in order to allow for the formation of a first capture antibody (or multiple antibody)-marker complex. The incubation can be carried out at a pH of from about 4.5 to about 10.0, at a temperature of from about 2° C. to about 45° C., and for a period from at least about one (1) minute to about eighteen (18) hours, from about 2-6 minutes, or from about 3-4 minutes.

After formation of the first/multiple capture antibody-marker complex, the complex is then contacted with at least one second detection antibody (under conditions which allow for the formation of a first/multiple antibody-marker second antibody complex). If the first antibody-marker complex is contacted with more than one detection antibody, then a first/multiple capture antibody-marker-multiple antibody detection complex is formed. As with first antibody, when the at least second (and subsequent) antibody is brought into contact with the first antibody-marker complex, a period of incubation under conditions similar to those described above is required for the formation of the first/multiple antibody-marker-second/multiple antibody complex. Preferably, at least one second antibody contains a detectable label. The detectable label can be bound to the at least one second antibody prior to, simultaneously with or after the formation of the first/multiple antibody-marker-second/multiple antibody complex. Any detectable label known in the art can be used.

### 8. KITS

Provided herein is a kit, which may be used for performing the methods described above. The kit may provide (1) at least one reagent capable of specifically binding the marker, i.e., CA19-9, CA125, or CUZD1, to quantify the levels of the marker, i.e., CA19-9, CA125, or CUZD1, in a biological sample isolated from a subject and (2) a reference standard indicating levels of the marker, wherein at least one reagent comprises at least one antibody capable of specifically binding the marker. The kit may further comprise at least one additional reagent capable of binding at least one additional biomarker selected from the group consisting of CA19-9, CA125, and CUZD1 to quantify the concentration of the at least one additional biomarker in the biological sample and a reference standard indicating the at least one additional biomarker in the biological sample, and a reference standard indicating a reference concentration of the at least one additional biomarker of sepsis or SIRS with infection or severe sepsis/septic shock, as discussed above.

The kit may comprise the antibody and a means for administering the antibody. The kit can further comprise instructions for using the kit and conducting the analysis, monitoring, or treatment.

The kit may also comprise one or more containers, such as vials or bottles, with each container containing a separate reagent. The kit may further comprise written instructions, which may describe how to perform or interpret an analysis, monitoring, treatment, or method described herein.

For example, the kit can comprise instructions for assaying the test sample for CA19-9, CA125, or CUZD1 by immunoassay, e.g., chemiluminescent microparticle immunoassay. The instructions can be in paper form or computer-readable form, such as a disk, CD, DVD, or the like. The antibody can be a CA19-9, CA125, or CUZD1 capture antibody and/or CA19-9, CA125, or CUZD1 detection antibody. Alternatively or additionally, the kit can comprise a calibrator or control, e.g., purified, and optionally lyophilized, (e.g., CA19-9, CA125, or CUZD1), and/or at least one container (e.g., tube, microtiter plates or strips, which can be already coated with an anti-CA19-9, CA125, or CUZD1 monoclonal antibody) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve or a reference standard for purposes of quantifying CA19-9, CA125, or CUZD1.

Any antibodies, which are provided in the kit, such as recombinant antibodies specific for CA19-9, CA125, or CUZD1, can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes or reagents for detecting the analyte. The antibodies, calibrators and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a blood sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution.

If desired, the kit can contain a solid phase, such as a magnetic particle, bead, test tube, microtiter plate, cuvette, membrane, scaffolding molecule, film, filter paper, a quartz crystal, disc or chip. The kit may also include a detectable label that can be or is conjugated to an antibody, such as an antibody functioning as a detection antibody. The detectable label can for example be a direct label, which may be an enzyme, oligonucleotide, nanoparticle, chemiluminophore, fluorophore, fluorescence quencher, chemiluminescence quencher, or biotin. Kits may optionally include any additional reagents needed for detecting the label.

If desired, the kit can further comprise one or more components, alone or in further combination with instructions, for assaying the test sample for another analyte, which can be a biomarker, such as a biomarker of cancer. Examples of analytes include, but are not limited to CA19-9, CA125, or CUZD1, and fragments of CA19-9, CA125, or CUZD1 as well other analytes and biomarkers discussed herein, or otherwise known in the art. In some embodiments one or more components for assaying a test sample for CA19-9, CA125, or CUZD1 enable the determination of the presence, amount or concentration of CA19-9, CA125, or CUZD1. A sample, such as a serum sample, can also be assayed for CA19-9, CA125, or CUZD1 using TOF-MS and an internal standard.

The kit (or components thereof), as well as the method of determining the concentration of CA19-9, CA125, or CUZD1 in a test sample by an immunoassay as described herein, can be adapted for use in a variety of automated and semi-automated systems (including those wherein the solid phase comprises a microparticle), as described, e.g., in U.S. Pat. Nos. 5,089,424 and 5,006,309, and as commercially marketed, e.g., by Abbott Laboratories (Abbott Park, Ill.) as ARCHITECT®.

Some of the differences between an automated or semi-automated system as compared to a non-automated system (e.g., ELISA) include the substrate to which the first specific binding partner (e.g., analyte antibody or capture antibody) is attached (which can impact sandwich formation and analyte reactivity), and the length and timing of the capture, detection and/or any optional wash steps. Whereas a non-automated format such as an ELISA may require a relatively longer incubation time with sample and capture reagent (e.g., about 2 hours), an automated or semi-automated format (e.g., ARCHITECT® and any successor platform, Abbott Laboratories) may have a relatively shorter incubation time (e.g., approximately 18 minutes for ARCHITECT®) Similarly, whereas a non-automated format such as an ELISA may incubate a detection antibody such as the conjugate reagent for a relatively longer incubation time (e.g., about 2 hours), an automated or semi-automated format (e.g., ARCHITECT® and any successor platform) may have a relatively shorter incubation time (e.g., approximately 4 minutes for the ARCHITECT® and any successor platform).

Other platforms available from Abbott Laboratories include, but are not limited to, AxSYM®, IMx® (see, e.g., U.S. Pat. No. 5,294,404, which is hereby incorporated by reference in its entirety), PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays Immunosensors and their methods of manufacture and operation in single-use test devices are described, for example in, U.S. Pat. No. 5,063,081, U.S. Pat. App. Pub. No. 2003/0170881, U.S. Pat. App. Pub. No. 2004/0018577, U.S. Pat. App. Pub. No. 2005/0054078, and U.S. Pat. App. Pub. No. 2006/0160164, which are incorporated in their entireties by reference for their teachings regarding same.

In particular, with regard to the adaptation of an assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an I-STAT® cartridge with a fluidics format suitable for immunoassay. On a portion of the wall of the sample-holding chamber of the cartridge there is a layer comprising the detection antibody labeled with alkaline phosphatase (or other label). Within the fluid pouch of the cartridge is an aqueous reagent that includes p-aminophenol phosphate.

In operation, a sample suspected of containing CA19-9, CA125, and/or CUZD1 is added to the holding chamber of the test cartridge and the cartridge is inserted into the I-STAT® reader. After the second antibody (detection antibody) has dissolved into the sample, a pump element within the cartridge forces the sample into a conduit containing the chip. Here it is oscillated to promote formation of the sandwich between the first capture antibody, CA19-9, CA125, and/or CUZD1, and the labeled second detection antibody. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of CA19-9, CA125, and/or CUZD1 in the sample by means of an embedded algorithm and factory-determined calibration curve.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

### 9. EXAMPLES

The present invention has multiple aspects, illustrated by the following non-limiting examples.

### Example 1

### Assay Formats

The levels of CA19-9 and CA125 were measured using the ARCHITECT® sandwich immunoassay. FIG. 1 shows an example of the ARCHITECT® sandwich immunoassay. An antibody coated on a microparticle captures the analyte of interest, a second antibody conjugated to acridinium binds to a second epitope on the analyte, the particles are separated from the label and a subsequent read is performed. The levels of CUZD1 were measured using enzyme-linked immunoassays (ELISA), which use an enzyme to quantitate the amount of protein biomarker in a serum sample. FIG. 2 shows a typical sandwich ELISA schematic: (1) a plate is coated with a capture antibody; (2) a sample is added and any antigen (protein biomarker for pancreatic cancer) present binds to the capture antibody; (3) a detecting antibody is added and binds to the antigen; (4) an enzyme-linked secondary antibody is added and binds to the detecting antibody; and (5) a substrate is added and converted by enzyme to a detectable form.

### Specimens

The 39 normal specimens were age matched serum specimens from normal individuals. The benign disease specimens (50 total) included 22 specimens with pancreatitis and various types of cysts and 28 specimens with pancreatic intraepithelial neoplasia (PanIN) and/or intraductal papillary mucinous neoplasm (IPMN) lesions (Table 1).

TABLE 1

| Specimen | n |
|---|---|
| Normal | 39 |
| Benign | 50 |
| Stage I | 22 |
| Stage II | 26 |
| Stage III | 26 |
| Stage IV | 26 |

### Biomarker Levels

The levels of CA19-9 in the samples were measured using the ARCHITECT® CA19-9 XR assay. FIG. 3 shows the ARCHITECT® CA19-9 XR dot plot results, which are summarized in Table 2.

TABLE 2
ARCHITECT ® CA19−9 XR

| | n | Mean | 95% CI | Min | 1st Quartile | Median | 95% CI |
|---|---|---|---|---|---|---|---|
| Normal | 39 | 11.70 | 6.76 to 16.64 | 2.00 | 2.14 | 5.71 | 3.36 to 10.29 |
| Benign | 50 | 19.97 | 8.20 to 31.74 | 2.00 | 3.95 | 8.49 | 6.33 to 12.59 |
| Stage I | 22 | 549.61 | −55.85 to 1155.06 | 2.00 | 58.15 | 120.72 | 58.92 to 448.16 |
| Stage II | 26 | 4009.29 | −603.49 to 8622.07 | 2.00 | 2.60 | 65.33 | 3.62 to 868.97 |
| Stage III | 26 | 3435.57 | −2499.50 to 9370.64 | 2.00 | 17.37 | 146.55 | 26.46 to 709.05 |
| Stage IV | 26 | 542737.57 | −507462.06 to 1592937.20 | 2.00 | 16.01 | 1393.72 | 18.51 to 17103.64 |

The levels of CA125 in the samples were measured using the ARCHITECT® CA-125 II assay. FIG. 4 shows the ARCHITECT® CA-125 II dot plot results, which are summarized in Table 3.

TABLE 3
ARCHITECT ® CA-125 II Dot Plot

| | n | Mean | 95% CI | Min | 1st Quartile | Median | 95% CI |
|---|---|---|---|---|---|---|---|
| Normal | 39 | 12.20 | 9.82 to 14.59 | 3.6 | 6.80 | 10.00 | 7.40 to 13.20 |
| Benign | 50 | 12.33 | 10.61 to 14.05 | 5.7 | 8.68 | 10.55 | 9.20 to 13.60 |
| Stage I | 22 | 18.01 | 13.04 to 22.99 | 6.0 | 9.45 | 15.35 | 9.50 to 22.10 |
| Stage II | 26 | 34.73 | 23.78 to 45.67 | 3.0 | 16.13 | 24.45 | 16.80 to 44.20 |
| Stage III | 26 | 57.25 | 34.32 to 80.17 | 10.5 | 17.51 | 43.15 | 19.70 to 57.60 |
| Stage IV | 26 | 118.13 | 58.28 to 177.97 | 10.1 | 23.13 | 67.60 | 31.90 to 107.30 |

The levels of CUZD1 in the samples were measured using ELISA Kit for CUB And Zona Pellucida Like Domains Protein 1 (CUZD1 ELISA) (USCN Life Sciences, Inc.). FIG. 5 shows the CUZD1 ELISA dot plot results, which are summarized in Table 4.

TABLE 4
CUZD1 ELISA

| | n | Mean | 95% CI | Min | 1st Quartile | Median | 95% CI |
|---|---|---|---|---|---|---|---|
| Normal | 38 | 1.86 | 1.40 to 2.33 | 1.25 | 1.25 | 1.25 | 1.25 to 1.63 |
| Benign | 50 | 1.68 | 1.38 to 1.98 | 1.25 | 1.25 | 1.25 | 1.25 to 1.40 |
| Stage I | 22 | 3.06 | 1.77 to 4.35 | 1.25 | 1.25 | 1.56 | 1.25 to 4.08 |
| Stage II | 26 | 6.82 | 4.27 to 9.37 | 1.25 | 2.23 | 3.93 | 2.55 to 8.04 |
| Stage III | 26 | 12.10 | 6.89 to 17.30 | 1.25 | 2.07 | 8.54 | 3.09 to 15.97 |
| Stage IV | 26 | 24.47 | 13.86 to 35.07 | 1.25 | 4.21 | 14.61 | 4.46 to 24.13 |

### Benign Versus Pancreatic Cancer Stages I and II Multivariate Models

Receiver operating characteristic (ROC) curves based on the measured levels of CA19-9, CA125, and CUZD1 were generated comparing benign with Stage I and Stage II pancreatic cancer, using the “Stage I and Stage II” as the positive level. The area under receiver operator curve (AUROC) was calculated using the software JMP software version 9.0.0. The following combinations were used as a panel: CA19-9, CA125, and CUZD1 (FIG. 6; AUROC=0.92375), CA19-9 and CUZD1 (FIG. 7; AUROC=0.89875), CA19-9 and CA125 (FIG. 8; AUROC=0.87104) and CA125 and CUZD1 (FIG. 9; AUROC=0.83479).

Tables 5 and 6 show a summary of the univariate and multivariate AUROC for the early detection of pancreatic cancer.

TABLE 5
Univariate AUROC: Early Detection of Pancreatic Cancer Study

| | | Benign vs | | |
|---|---|---|---|---|
| Manufacturer/ | | Stage I & | Benign vs | Non-Cancer |
| System Format | Biomarker | II | Cancer | vs Cancer |
| USCN Life | CUZD1 | 0.79 | 0.82 | 0.81 |
| Sciences/ELISA | | | | |
| Abbott Laboratories/ | CA 19-9 XR | 0.77 | 0.80 | 0.81 |
| ARCHITECT ® | | | | |
| Abbott Laboratories/ | CA 125 II | 0.76 | 0.83 | 0.84 |
| ARCHITECT ® | | | | |
| Abbott Laboratories/ | CA15-3 | 0.64 | 0.68 | 0.68 |
| ARCHITECT ® | | | | |
| USCN Life | COL6a3 | 0.64 | 0.66 | 0.66 |
| Sciences/ELISA | | | | |
| Abbott Laboratories/ | TIMP-1 | 0.63 | 0.69 | 0.70 |
| ARCHITECT ® | | | | |
| Abbott Laboratories/ | CEA | 0.62 | 0.66 | 0.67 |
| ARCHITECT ® | | | | |
| USCN Life | AGR2 | 0.60 | 0.68 | 0.67 |
| Sciences/ELISA | | | | |
| USCN Life | REG1b | 0.59 | 0.65 | 0.68 |
| Sciences/ELISA | | | | |
| USCN Life | SYCN | 0.57 | 0.53 | 0.51 |
| Sciences/ELISA | | | | |
| IBL/ELISA | LRG1 | 0.57 | 0.63 | 0.62 |
| Abbott Laboratories/ | NGAL | 0.52 | 0.51 | 0.56 |
| ARCHITECT ® | | | | |
| Abbott Laboratories/ | HE4 | 0.51 | 0.55 | 0.56 |
| ARCHITECT ® | | | | |

TABLE 6
Multivariate AUROC: Early Detection of Pancreatic Cancer Study

| | | Benign vs | | |
|---|---|---|---|---|
| Manufacturer/ | | Stage | Benign vs | Non-Cancer |
| System Format | Biomarker | I & II | Cancer | vs Cancer |
| Abbott Laboratories/ | CA 19-9 XR | 0.92 | 0.96 | 0.96 |
| ARCHITECT ® | | | | |
| Abbott Laboratories/ | CA 125 II | | | |
| ARCHITECT ® | | | | |
| USCN Life | CUZD1 | | | |
| Sciences/ELISA | | | | |
| Abbott Laboratories/ | CA 19-9 XR | 0.90 | 0.94 | 0.94 |
| ARCHITECT ® | | | | |
| Abbott Laboratories/ | CUZD1 | | | |
| ARCHITECT ® | | | | |
| Abbott Laboratories/ | CA 19-9 XR | 0.87 | 0.89 | 0.90 |
| ARCHITECT ® | | | | |
| Abbott Laboratories/ | CA 125 II | | | |
| ARCHITECT ® | | | | |
| Abbott Laboratories/ | CA 125 II | 0.83 | 0.92 | 0.92 |
| ARCHITECT ® | | | | |
| Abbott Laboratories/ | CUZD1 | | | |
| ARCHITECT ® | | | | |

### Validation Evaluation

Validation evaluation was performed using the resubstitution, split-sample, leave-one-out, K-fold cross, and bootstrapping validation models.

In the resubstitution validation model, measures of model performance were computed from the same data that was used for model fitting, also known as “double-dipping” since the data was used twice, once for fitting and once for predicting. Measures of model performance tended to reflect optimistic predictions because they indicated better accuracy than the actual model would allow in practice. Using forward stepwise methods, the model that met the statistical inclusion criteria included CUZD1 and CA 19-9 XR. Resubstitution often results in an overly fitted, overly optimistic model. With this model, the AUC was 0.84. The bias seen in resubstitution can often be reduced by using the split sample procedure.

In the split-sample validation model, data was split into a training dataset and test dataset. The training dataset was used to fit the model and the performance of this model was evaluated using the test dataset. Model performance was often less biased. The predictive model was built on the training dataset and the estimates for the model discrimination were obtained using the test dataset. In order to estimate the model discrimination, the predicted probabilities obtained from the fitted model were used as the marker values, resulting in a corrected AUC of 0.80.

The leave-one-out validation model is a sample reuse method that increases the efficiency by repeated use of observed data points and estimating the model performance with smaller variance, but has an increase in bias since each observation is used more than once. In this method, one observation of the dataset was left out and a model was built on the remaining observations. The model was then used to predict the left-out sample. This process was repeated for each observation, resulting in a new dataset with a predicated probability for each observation that was used to construct the cross-validated discrimination measure. Similar results were seen with this validation method. An AUC of 0.84 was corrected to 0.81 using the cross-validated sample, producing a model with CUZD1 and CA 125 II.

The K-fold cross validation model is another sample reuse sample method that breaks the data into k randomly chosen segments, wherein the analysis is repeated k times. For each of the analyses, one of the k segments was used as the test dataset and the other k-1 segments were used as the training datasets. One prediction for each observation formed the basis of the k-fold cross validated discrimination measure. Using a 5-fold cross validation method, one of the 5 segments was left out as the test set and the remaining 4 sets were used for training, producing 5. Each one was optimally built using CUZD1 and CA 125 II, with AUCs ranging from 0.83 to 0.91. One model added CA 19-9 XR to the model as well. The final cross-validated discrimination measure resulted in an AUC of 0.81.

The Bootstrapping validation model is another sample reuse method that can result in lower variances as a result of forming B bootstrap samples, where B is typically greater than 100. B bootstrap samples were formed and used as the training sample. The original data and the bootstrap sample were then used as test samples. The difference in the discrimination measure estimated from the original sample and from the bootstrap method was a measure of optimism. This optimism value was subtracted from the resubstitution measure of discrimination to produce an optimism-corrected measure of discrimination. 100 bootstraps were performed. The measure of optimism was subtracted from the resubstitution AUC to produce an optimism-corrected AUC. With this method, a validated measure of discrimination was calculated as AUC equal to 0.73 when all models were allowed to be selected into the model. Another model was built whereupon only CA 125 II was considered. The corrected AUC remained the same as the resubstitution AUC was 0.76.

As shown in Table 7, all of the markers were considered unless otherwise noted in the text. The best model was selected using forward stepwise selection methodology.

TABLE 7

| | | Training | | | | Test Validation | | |
|---|---|---|---|---|---|---|---|---|
| | | | 95% CI | | | | 95% CI | |
| | | | Lo- | Up- | | | Lo- | Up- |
| Method | Model | OR | wer | per | AUC | AUC | wer | per |
| Resubsti- | CUZD1 | 1.78 | 1.22 | 2.59 | 0.84 | | | |
| tution | CA125 II | 1.10 | 1.03 | 1.17 | | | | |
| Split-Sample | CUZD1 | 1.96 | 1.21 | 3.16 | 0.78 | 0.80 | 0.65 | 0.95 |
| Leave One | CUZD1 | 1.78 | 1.22 | 2.60 | 0.84 | 0.81 | 0.72 | 0.90 |
| Out | CA125 II | 1.10 | 1.03 | 1.17 | | | | |
| K- Fold | CUZD1 | 1.62 | 1.09 | 2.41 | 0.86 | | | |
| | CA125 II | 1.17 | 1.05 | 1.30 | | | | |
| | CUZD1 | 2.17 | 1.21 | 3.88 | 0.91 | | | |
| | CA125 II | 1.09 | 1.00 | 1.18 | | | | |
| | CA19-9 | 1.01 | 1.00 | 1.02 | | | | |
| | XR | | | | | | | |
| | CUZD1 | 1.98 | 1.23 | 3.18 | 0.84 | | | |
| | CA125 II | 1.07 | 1.01 | 1.14 | | | | |
| | CUZD1 | 1.81 | 1.19 | 2.74 | 0.86 | | | |
| | CA125 II | 1.11 | 1.03 | 1.20 | | | | |
| | CUZD1 | 1.63 | 1.14 | 2.33 | 0.83 | | | |
| | CA125 II | 1.10 | 1.02 | 1.18 | | 0.81 | 0.73 | 0.91 |
| Boot- | Various | | | | 0.84 | 0.80 | | |
| strapping | | | | | | | | |

Data from the Effects likelihood ratio test within Fit Model in JMP Version 9.0.0 was generated for the different markers. As shown in Table 8, the correlation between CA19-9 XR and CA125 II (0.1019) and between CA19-9 XR and CUZD1 (0.1100) indicated that these biomarkers were independent and did not correlate when discriminating between benign versus stages 1 and 2 pancreatic cancer. There was only a moderate correlation between CA125 II and CUZD1 (0.5018).

TABLE 8
Multivariable Statistical Evaluation: Correlations

| Multivariate | | | | | | | |
|---|---|---|---|---|---|---|---|
| Correlations | NGAL | HE4 | CEA | CA 19-9 XR | CA 125 II | AGR2 | LRG1 |
| NGAL | 1.0000 | 0.7310 | −0.0751 | −0.0741 | 0.0550 | −0.0508 | 0.5463 |
| HE4 | 0.7310 | 1.0000 | −0.0088 | −0.0633 | 0.1329 | −0.0052 | 0.4195 |
| CEA | −0.0751 | −0.0088 | 1.0000 | 0.8503 | 0.0807 | 0.9195 | −0.0410 |
| CA 19-9 XR | −0.0741 | −0.0633 | 0.8503 | 1.0000 | 0.1019 | 0.7767 | −0.0635 |
| CA 125 II | 0.0550 | 0.1329 | 0.0807 | 0.1019 | 1.0000 | 0.0593 | 0.2154 |
| AGR2 | −0.0508 | −0.0052 | 0.9195 | 0.7767 | 0.0593 | 1.0000 | −0.0067 |
| LRG1 | 0.5463 | 0.4195 | −0.0410 | −0.0635 | 0.2154 | −0.0067 | 1.0000 |
| TIMP-1 | −0.0306 | 0.0205 | 0.2479 | 0.2007 | 0.1317 | 0.1538 | 0.0977 |
| CA15-3 | 0.0983 | 0.2560 | 0.1351 | 0.2064 | 0.1313 | 0.1064 | 0.0307 |
| CUZD1 | 0.0760 | 0.1461 | −0.0051 | 0.1100 | 0.5018 | −0.0151 | 0.1500 |
| REG1b | 0.1240 | 0.1061 | 0.0991 | −0.0004 | 0.1134 | 0.0331 | 0.2457 |
| SYCN | 0.0211 | 0.0480 | 0.1378 | 0.2619 | 0.0652 | 0.0642 | −0.0648 |
| COL6a3 | 0.0206 | 0.0240 | 0.1985 | 0.1259 | 0.0626 | 0.1273 | 0.0749 |
| | Multivariate | | | | | | |
| | Correlations | TIMP-1 | CA15-3 | CUZD1 | REG1b | SYCN | COL6a3 |
| | NGAL | −0.0306 | 0.0983 | 0.0760 | 0.1240 | 0.0211 | 0.0206 |
| | HE4 | 0.0205 | 0.2560 | 0.1461 | 0.1061 | 0.0480 | 0.0240 |
| | CEA | 0.2479 | 0.1351 | −0.0051 | 0.0991 | 0.1378 | 0.1985 |
| | CA 19-9 XR | 0.2007 | 0.2064 | 0.1100 | −0.0004 | 0.2619 | 0.1259 |
| | CA 125 II | 0.1317 | 0.1313 | 0.5018 | 0.1134 | 0.0652 | 0.0626 |
| | AGR2 | 0.1538 | 0.1064 | −0.0151 | 0.0331 | 0.0642 | 0.1273 |
| | LRG1 | 0.0977 | 0.0307 | 0.1500 | 0.2457 | −0.0648 | 0.0749 |
| | TIMP-1 | 1.0000 | 0.1714 | 0.3068 | 0.4636 | 0.3737 | 0.6865 |
| | CA15-3 | 0.1714 | 1.0000 | 0.1467 | 0.0678 | 0.1224 | 0.1068 |
| | CUZD1 | 0.3068 | 0.1467 | 1.0000 | 0.2222 | 0.3974 | 0.2670 |
| | REG1b | 0.4636 | 0.0678 | 0.2222 | 1.0000 | 0.0430 | 0.4296 |
| | SYCN | 0.3737 | 0.1224 | 0.3974 | 0.0430 | 1.0000 | 0.2697 |
| | COL6a3 | 0.6865 | 0.1068 | 0.2670 | 0.4296 | 0.2697 | 1.0000 |

As described above, the combination of CA19-9 XR, CA125 II, and CUZD1 biomarkers discriminated between benign disease and early stage I and II cancers. In the univariate analysis, CUZD1, CA125 II, and CA19-9 XR had an AUROC of 0.79, 0.76, and 0.77, respectively. The multivariate analysis yielded three biomarkers that were statistically significant in the Fit Model function using the software JMP version 9.0.0. When CA19-9 XR, CA125 II, and CUZD1 were combined, the AUROC significantly increased to 0.92 for benign versus stage I and II pancreatic cancer. This performance was significantly better than CA19-9 alone and useful for the early detection of pancreatic cancer.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A method for identifying and treating a subject having or at risk of having pancreatic cancer, the method comprising the steps of:
(a) obtaining a biological sample from the subject;
(b) determining the levels of carbohydrate antigen 19-9 (CA19-9), carbohydrate antigen 125 (CA125), and CUB and zona pellucida-like domains 1 (CUZD1) in the biological sample from the subject;
(c) comparing the levels of CA19-9, CA125, and CUZD1 in the biological sample to reference levels of CA19-9, CA125, and CUZD1;
(d) identifying the subject as having pancreatic cancer or at risk of having pancreatic cancer if the levels of CA19-9, CA125, and CUZD1 in the biological sample are greater than the reference levels of CA19-9, CA125, and CUZD1; and
(e) administering a therapy against pancreatic cancer to the subject identified as having pancreatic cancer or a means by which to monitor pancreatic status in said subject identified as at risk of having pancreatic cancer.

2. The method of claim 1, further comprising a step of administering a means for confirming the subject has pancreatic cancer by performing a biopsy, measuring pancreatic function, or imaging with MRI, PET scan, CT scan, ultrasound, endoscopic ultrasound (EUS), endoscopic retrograde cholangiopancreatography (ERCP), EUS cholangiopancreatography, MRI/magnetic resonance cholangiopancreatography (MRI/MRCP) or laparoscopy.

3. The method of claim 1, wherein the reference levels of CA19-9, CA125, and CUZD1 are the levels of CA19-9, CA125, and CUZD1 in a control sample.

4. The method of claim 3, wherein the control sample is a sample from a benign or healthy patient.

5. The method of claim 1, wherein the reference levels of CA19-9, CA125, and CUZD1 are the CA19-9, CA125, and CUZD1 cutoff values determined by a receiver operating curve (ROC) analysis from biological samples of a patient group.

6. The method of claim 1, wherein the reference levels of CA19-9, CA125, and CUZD1 are the CA19-9, CA125, and CUZD1 cutoff values determined by a quartile analysis of biological samples of a patient group.

7. The method of claim 6, wherein the CA19-9, CA125, and CUZD1 cutoff values are about 20 ng/mL in serum for CA19-9, about 15 ng/mL for CA125, and about 1.5 ng/mL in serum for CUZD1.

8. The method of claim 7, wherein CA19-9, CA125, and CUZD1 levels in the biological sample above the cutoffs indicate that the subject has early stage pancreatic cancer or late stage pancreatic cancer.

9. The method of claim 8, wherein the subject has stage I pancreatic cancer.

10. The method of claim 6, wherein the CA19-9, CA125, and CUZD1 cutoff values are about 20 ng/mL in serum for CA19-9, about 15 ng/mL for CA125, and about 1.5 ng/mL in serum for CUZD1.

11. The method of claim 10, wherein sample levels about the cutoffs indicate that the subject has late stage pancreatic cancer.

12. The method of claim 1, further comprising determining the level of at least one additional biomarker of pancreatic cancer in the biological sample, and comparing the level of the at least one additional biomarker of pancreatic cancer to a reference concentration value for the at least one biomarker of pancreatic cancer.

13. The method of claim 12, wherein the at least one additional biomarker of pancreatic cancer is selected from the group consisting of CEA, HE4, NGAL, TIMP-1, CA15-3, AGR2, LRG1, COL6A3, REG1B and SYCN.

14. The method of claim 1, wherein the subject is a human.

15. The method of claim 1, wherein the biological sample of a subject is selected from a tissue sample, bodily fluid, whole blood, plasma, serum, urine, bronchoalveolar lavage fluid, and a cell culture suspension or fraction thereof.

16. The method of claim 1, wherein the biological sample of a subject is blood plasma or blood serum.

17. The method of claim 1, wherein determining the levels of CA19-9, CA125, and CUZD1 comprises an immunological method with molecules binding to CA19-9, CA125, and CUZD1.

18. The method of claim 17, wherein the molecules binding to CA19-9, CA125, and CUZD1 comprises at least one antibody capable of specifically binding CA19-9, CA125, or CUZD1.

19. The method of claim 17, wherein the immunological method comprises:
(a) measuring the levels of CA19-9 by:
(i) contacting the test sample with at least one capture antibody, wherein the capture antibody binds to an epitope on CA19-9 or a fragment of CA19-9 to form a capture antibody-CA19-9 antigen complex;
(ii) contacting the capture antibody-CA19-9 antigen complex with at least one detection antibody comprising a detectable label, wherein the detection antibody binds to an epitope on CA19-9 that is not bound by the capture antibody and forms a capture antibody-CA19-9 antigen-detection antibody complex; and
(iii) determining the CA19-9 levels in the test sample based on the signal generated by the detectable label in the capture antibody-CA19-9 antigen-detection antibody complex formed in (a)(ii);
(b) measuring the levels of CA125 by:
(i) contacting the test sample with at least one capture antibody, wherein the capture antibody binds to an epitope on CA125 or a fragment of CA125 to form a capture antibody-CA125 antigen complex;
(ii) contacting the capture antibody-CA125 antigen complex with at least one detection antibody comprising a detectable label, wherein the detection antibody binds to an epitope on CA125 that is not bound by the capture antibody and forms a capture antibody-CA125 antigen-detection antibody complex; and
(iii) determining the CA125 levels in the test sample based on the signal generated by the detectable label in the capture antibody-CA125 antigen-detection antibody complex formed in (b)(ii); and
(c) measuring the levels of CUZD1 by:
(i) contacting the test sample with at least one capture antibody, wherein the capture antibody binds to an epitope on CUZD1 or a fragment of CUZD1 to form a capture antibody-CUZD1 antigen complex;
(ii) contacting the capture antibody-CUZD1 antigen complex with at least one detection antibody comprising a detectable label, wherein the detection antibody binds to an epitope on CUZD1 that is not bound by the capture antibody and forms a capture antibody-CUZD1 antigen-detection antibody complex; and
(iii) determining the CUZD1 levels in the test sample based on the signal generated by the detectable label in the capture antibody-CUZD1 antigen-detection antibody complex formed in (c)(ii).

20. The method of claim 1, wherein the therapy against pancreatic cancer comprises administering at least one of surgery, radiation therapy, chemotherapy, and targeted therapy to the subject.

21. The method of claim 1, wherein the means by which to monitor pancreatic status comprises at least one of determining the levels of at least one biomarker of pancreatic cancer at periodic intervals, performing a biopsy, measuring pancreatic function, or imaging with MRI, PET scan, CT scan, ultrasound, endoscopic ultrasound (EUS), endoscopic retrograde cholangiopancreatography (ERCP), EUS cholangiopancreatography, MRI/magnetic resonance cholangiopancreatography (MRI/MRCP) or laparoscopy.

22. A method for the diagnosis, prognosis and/or risk stratification of pancreatic cancer in a subject having or suspected of pancreatic cancer, the method comprising the step of detecting increased levels of carbohydrate antigen 19-9 (CA19-9), carbohydrate antigen 125 (CA125), and CUB and zona pellucida-like domains 1 (CUZD1) in the subject relative to a control subject not having pancreatic cancer.

23. The method of claim 22, further comprising a step of administering a means for confirming the diagnosis, prognosis and/or risk stratification of pancreatic cancer in a subject by performing a biopsy, measuring pancreatic function, or imaging with MRI, PET scan, CT scan, ultrasound, endoscopic ultrasound (EUS), endoscopic retrograde cholangiopancreatography (ERCP), EUS cholangiopancreatography, MRI/magnetic resonance cholangiopancreatography (MRI/MRCP) or laparoscopy.

24. A method of providing a diagnosis of a subject having pancreatic cancer, the method comprising the steps of:
(a) obtaining a biological sample comprising blood from the subject;
(b) determining the level of carbohydrate antigen 19-9 (CA19-9), carbohydrate antigen 125 (CA125), and CUB and zona pellucida-like domains 1 (CUZD1) in the biological sample from the subject;
(c) comparing the levels of CA19-9, CA125, and CUZD1 in the biological sample to reference levels of CA19-9, CA125, and CUZD1; and
(d) providing a diagnosis of a subject having pancreatic cancer if the levels of CA19-9, CA125, and CUZD1 in the biological sample are greater than the reference levels of CA19-9, CA125, and CUZD1.

25. The method of claim 24, further comprising a step of administering a means for confirming the subject has pancreatic cancer by performing a biopsy, measuring pancreatic function, or imaging with MRI, PET scan, CT scan, ultrasound, endoscopic ultrasound (EUS), endoscopic retrograde cholangiopancreatography (ERCP), EUS cholangiopancreatography, MRI/magnetic resonance cholangiopancreatography (MRI/MRCP) or laparoscopy.

26. A method of determining whether a subject is suffering from early or late stage pancreatic cancer, the method comprising the steps of:
(a) obtaining a biological sample comprising blood from the subject;
(b) determining the level of carbohydrate antigen 19-9 (CA19-9), carbohydrate antigen 125 (CA125), and CUB and zona pellucida-like domains 1 (CUZD1) in the biological sample from the subject;
(c) comparing the levels of CA19-9, CA125, and CUZD1 in the biological sample to reference levels of CA19-9, CA125, and CUZD1; and
(d) providing a diagnosis of a subject suffering from early stage or late stage pancreatic cancer if the levels of CA19-9, CA125, and CUZD1 in the biological sample are greater than the reference levels of CA19-9, CA125, and CUZD1.

27. The method of claim 26, further comprising a step of administering a means for confirming the subject is suffering from early stage or late stage pancreatic cancer by performing a biopsy, measuring pancreatic function, or imaging with MRI, PET scan, CT scan, ultrasound, endoscopic ultrasound (EUS), endoscopic retrograde cholangiopancreatography (ERCP), EUS cholangiopancreatography, MRI/magnetic resonance cholangiopancreatography (MRI/MRCP) or laparoscopy.

28. The method of claim 24 or 26, wherein the reference levels of CA19-9, CA125, and CUZD1 are the levels of CA19-9, CA125, and CUZD1 in a control sample.

29. The method of claim 28, wherein the control sample is a sample from benign or normal patients.

30. The method of claim 24 or 26, wherein the reference levels of CA19-9, CA125, and CUZD1 are the CA19-9, CA125, and CUZD1 cutoff values determined by a receiver operating curve (ROC) analysis from biological samples of a patient group.

31. The method of claim 24 or 26, wherein the reference levels of CA19-9, CA125, and CUZD1 are the CA19-9, CA125, and CUZD1 cutoff values determined by a quartile analysis of biological samples of a patient group.

32. The method of claim 31, wherein the CA19-9, CA125, and CUZD1 cutoff values are about 20 ng/mL in serum for CA19-9, about 15 ng/mL for CA125, and about 1.5 ng/mL in serum for CUZD1.

33. The method of claim 32, wherein CA19-9, CA125, and CUZD1 levels in the biological sample above the cutoffs indicate that the subject has early stage pancreatic cancer or late stage pancreatic cancer.

34. The method of claim 33, wherein the subject has stage I pancreatic cancer.

35. The method of claim 31, wherein the CA19-9, CA125, and CUZD1 cutoff values are about 20 ng/mL in serum for CA19-9, about 15 ng/mL for CA125, and about 1.5 ng/mL in serum for CUZD1.

36. The method of claim 35, wherein sample levels about the cutoffs indicate that the subject has late stage pancreatic cancer.

37. The method of any one of claim 22, 24 or 26, further comprising determining the level of at least one additional biomarker of pancreatic cancer in the biological sample, and comparing the level of the at least one additional biomarker of pancreatic cancer to a reference concentration value for the at least one biomarker of pancreatic cancer.

38. The method of claim 37, wherein the at least one additional biomarker of pancreatic cancer is selected from the group consisting of CEA, HE4, NGAL, TIMP-1, CA15-3, AGR2, LRG1, COL6A3, REG1B and SYCN.

39. The method of any one of claim 22, 24 or 26, wherein the subject is a human.

40. The method of any one of claim 22, 24 or 26, wherein the biological sample of a subject is selected from a tissue sample, bodily fluid, whole blood, plasma, serum, urine, bronchoalveolar lavage fluid, and a cell culture suspension or fraction thereof.

41. The method of any one of claim 22, 24 or 26, wherein the biological sample of a subject is blood plasma or blood serum.

42. The method of any one of claim 22, 24 or 26, wherein determining the levels of CA19-9, CA125, and CUZD1 comprises an immunological method with molecules binding to CA19-9, CA125, and CUZD1.

43. The method of claim 42, wherein the molecules binding to CA19-9, CA125, and CUZD1 comprises at least one antibody capable of specifically binding CA19-9, CA125, or CUZD1.

44. The method of claim 42, wherein the immunological method comprises:
(a) measuring the levels of CA19-9 by:
(i) contacting the test sample with at least one capture antibody, wherein the capture antibody binds to an epitope on CA19-9 or a fragment of CA19-9 to form a capture antibody-CA19-9 antigen complex;
(ii) contacting the capture antibody-CA19-9 antigen complex with at least one detection antibody comprising a detectable label, wherein the detection antibody binds to an epitope on CA19-9 that is not bound by the capture antibody and forms a capture antibody-CA19-9 antigen-detection antibody complex; and
(iii) determining the CA19-9 levels in the test sample based on the signal generated by the detectable label in the capture antibody-CA19-9 antigen-detection antibody complex formed in (a)(ii);
(b) measuring the levels of CA125 by:
(i) contacting the test sample with at least one capture antibody, wherein the capture antibody binds to an epitope on CA125 or a fragment of CA125 to form a capture antibody-CA125 antigen complex;
(ii) contacting the capture antibody-CA125 antigen complex with at least one detection antibody comprising a detectable label, wherein the detection antibody binds to an epitope on CA125 that is not bound by the capture antibody and forms a capture antibody-CA125 antigen-detection antibody complex; and
(iii) determining the CA125 levels in the test sample based on the signal generated by the detectable label in the capture antibody-CA125 antigen-detection antibody complex formed in (b)(ii); and
(c) measuring the levels of CUZD1 by:
(i) contacting the test sample with at least one capture antibody, wherein the capture antibody binds to an epitope on CUZD1 or a fragment of CUZD1 to form a capture antibody-CUZD1 antigen complex;
(ii) contacting the capture antibody-CUZD1 antigen complex with at least one detection antibody comprising a detectable label, wherein the detection antibody binds to an epitope on CUZD1 that is not bound by the capture antibody and forms a capture antibody-CUZD1 antigen-detection antibody complex; and
(iii) determining the CUZD1 levels in the test sample based on the signal generated by the detectable label in the capture antibody-CUZD1 antigen-detection antibody complex formed in (c)(ii).

45. The method of any one of claim 22, 24 or 26, further comprising a step of administering at least one of a therapy against pancreatic cancer to the subject or a means by which to monitor pancreatic status in the subject.

46. A method of monitoring the progression of pancreatic cancer in a subject, the method comprising:
(a) obtaining a biological sample from the subject;
(b) determining the levels of carbohydrate antigen 19-9 (CA19-9), carbohydrate antigen 125 (CA125), and CUB and zona pellucida-like domains 1 (CUZD1) in the biological sample from the subject;
(c) correlating the levels of CA19-9, CA125, and CUZD1 with progression of pancreatic cancer in the subject, wherein if the levels of CA19-9, CA125, and CUZD1 are higher as compared to the levels of CA19-9, CA125, and CUZD1 in an earlier biological sample from the subject, the subject is identified as having progression of pancreatic cancer.

47. A method of determining the stage of pancreatic cancer in a subject, the method comprising the steps of:
(a) obtaining a biological sample comprising blood from the subject;
(b) determining the level of carbohydrate antigen 19-9 (CA19-9), carbohydrate antigen 125 (CA125), and CUB and zona pellucida-like domains 1 (CUZD1) in the biological sample from the subject;
(c) comparing the levels of CA19-9, CA125, and CUZD1 in the biological sample to first reference levels of CA19-9, CA125, and CUZD1 and second reference levels of CA19-9, CA125, and CUZD1; and
(d) correlating the levels of CA19-9, CA125, and CUZD1 in the biological sample with the stage of pancreatic cancer in the subject, wherein if the levels of CA19-9, CA125, and CUZD1 are greater than the first reference levels of CA19-9, CA125, and CUZD1 but less than the second reference levels of CA19-9, CA125, and CUZD1, the subject is determined to have early stage pancreatic cancer, and wherein if the levels of CA19-9, CA125, and CUZD1 are greater than the first reference levels of CA19-9, CA125, and CUZD1 and the second reference levels of CA19-9, CA125, and CUZD1, the subject is determined to have late stage pancreatic cancer.

48. The method of claim 47, further comprising a step of administering at least one of a therapy against pancreatic cancer to the subject or a means by which to monitor pancreatic status in the subject.

49. A kit for performing the method of claim 1, the kit comprising:
(a) at least one reagent capable of specifically binding CA19-9, CA125, or CUZD1 to quantify the levels of CA19-9, CA125, or CUZD1 in the biological sample of a subject; and
(b) a reference standard indicating reference levels of CA19-9, CA125, and CUZD1.

50. The kit of claim 49, wherein the at least one reagent comprises at least one antibody capable of specifically binding CA19-9, CA125, or CUZD1.

51. The kit of claim 49, further comprising at least one additional reagent capable of binding at least one additional biomarker of CA19-9, CA125, and CUZD1 in the biological sample to quantify the concentration of the at least one additional biomarker in the biological sample, and a reference standard indicating a reference concentration of the at least one additional biomarker of CA19-9, CA125, and CUZD1 in the biological sample.
